(19) United States
(12) Patent Application Publication
(10) Pub. No.: US 2025/0161399 A1
(43) Pub. Date: May 22, 2025

(54) TARGETING SLC46A2-MEDIATED MUROPEPTIDE TRANSPORT TO TREAT PSORIASIS

(71) Applicant: University of Massachusetts, Boston, MA (US)

(72) Inventors: Neal SILVERMAN, Boxborough, MA (US); Ravi BHARADWAJ, Worchester, MA (US); Megan ORZALLI, Jamacia Plain, MA (US)

(21) Appl. No.: 18/700,305

(22) PCT Filed: Oct. 12, 2022
(86) PCT No.: PCT/US22/46425

§ 371 Date: Apr. 11, 2024

Related U.S. Application Data
(60) Provisional application No. 63/358,404, filed on Jul. 5, 2022.
(60) Provisional application No. 63/254,756, filed on Oct. 12, 2021.

(51) Int. Cl. A61K 38/05; A61K 9/00; A61K 31/519; A61P 17/06; A61P 29/00

(52) U.S. Cl. CPC A61K 38/05; A61K 9/0014; A61K 31/519; A61P 17/06; A61P 29/00

(57) ABSTRACT

Methods for treating inflammatory skin diseases involving inhibition of solute carrier family 46 member 2 (SLC46A2) and/or solute carrier family 46 member 3 (SLC46A3).

### CLAIM OF PRIORITY

This application claims the benefit of U.S. Provisional Patent Application No. 63/254,756, filed on Oct. 12, 2021, and 63/358,404, filed on Jul. 5, 2022, each of which is incorporated by reference herein in its entirety.

### FEDERALLY SPONSORED RESEARCH OR DEVELOPMENT

This invention was made with government support under Grant No. AI060025 awarded by the National Institutes of Health. The government has certain rights in the invention.

### FIELD OF THE INVENTION

The subject matter disclosed herein generally relates to methods and compositions for treating inflammatory skin diseases.

### BACKGROUND OF THE INVENTION

Small fragments of peptidoglycan (PGN), the major constituent of the bacterial cell wall, are often shed into the environment. These fragments (also referred to as muropeptides) readily enter the cytosol of some cells where they initiate inflammatory and innate immune responses. However, the mechanisms by which muropeptides trigger inflammatory and immune responses is unclear.

### SUMMARY OF THE INVENTION

The present disclosure is based, at least in part, on the surprising discovery that mice lacking solute carrier family 46 member 2 (SLC46A2), a proton coupled folate transporter, were resistant to imiquimod (IMQ)-induced and muropeptide-induced skin inflammation. It was also shown that SLC46A2 was specific for the inflammatory response to NOD1 ligands such as γ-D-Glu-mDAP (iE-DAP) while SLC46 member 3 (SLC46A3) was specific for the inflammatory response to NOD2 ligands such as muramyl dipeptide (MDP).

Accordingly, aspects of the present disclosure provide methods for treating inflammatory skin diseases comprising administering to a subject in need thereof an effective amount of an inhibitor of SLC46A2 and/or an inhibitor of SLC46A3.

In some embodiments, the inhibitor is selected from the group consisting of a small molecule inhibitor, a peptide inhibitor, an antibody or antigen binding fragment thereof, and an agent that inhibits expression of SLC46A2 and/or SLC46A3.

In some embodiments, the agent that inhibits expression of SLC46A2 and/or SLC46A3 is selected from the group consisting of short interfering nucleic acid (siNA), short interfering RNA (siRNA), double-stranded RNA (dsRNA), micro-RNA (miRNA), and short hairpin RNA (shRNA).

In some embodiments, the inhibitor is formulated for topical administration. In some embodiments, the inhibitor is formulated in a pharmaceutical composition, which further comprises a pharmaceutically acceptable carrier.

In some embodiments, the subject is a human patient having or at risk for having an inflammatory skin disease. In some embodiments, the inflammatory skin disease is psoriasis, skin infections, dermatitis, acne, rosacea, cutaneous lupus erythematosus, lichen planus, cutaneous dermatomyositis, pityriasis rubra pilaris, and hidradenitis suppurativa.

In some embodiments, the inhibitor is administered topically. In some embodiments, the inhibitor is administered systemically.

In some embodiments, methods described herein further comprise administering to the subject an additional therapeutic agent. In some embodiments, the additional therapeutic agent is an anti-inflammatory agent or an immunosuppressant. In some embodiments, the anti-inflammatory agent is methotrexate. In some embodiments, the inhibitor and the additional therapeutic agent are formulated in a single formulation.

### DETAILED DESCRIPTION OF THE DRAWINGS

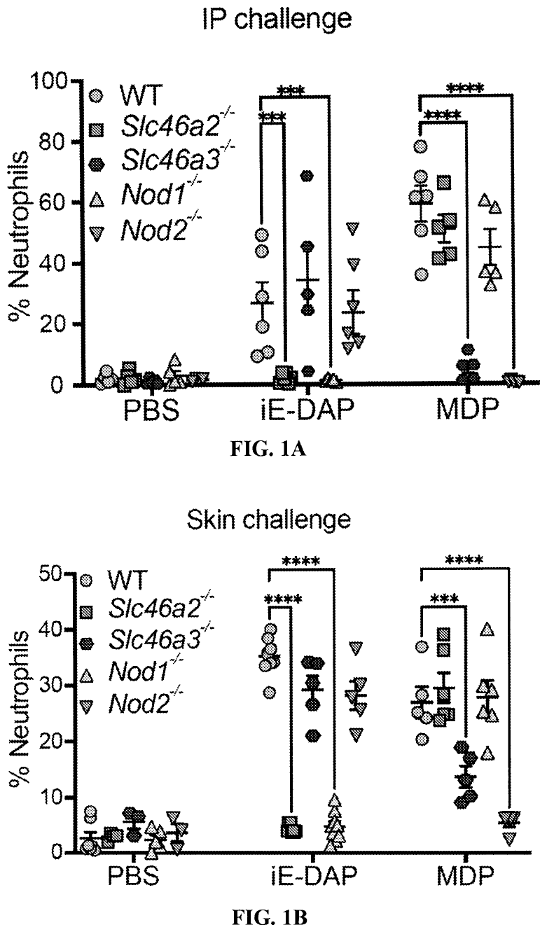

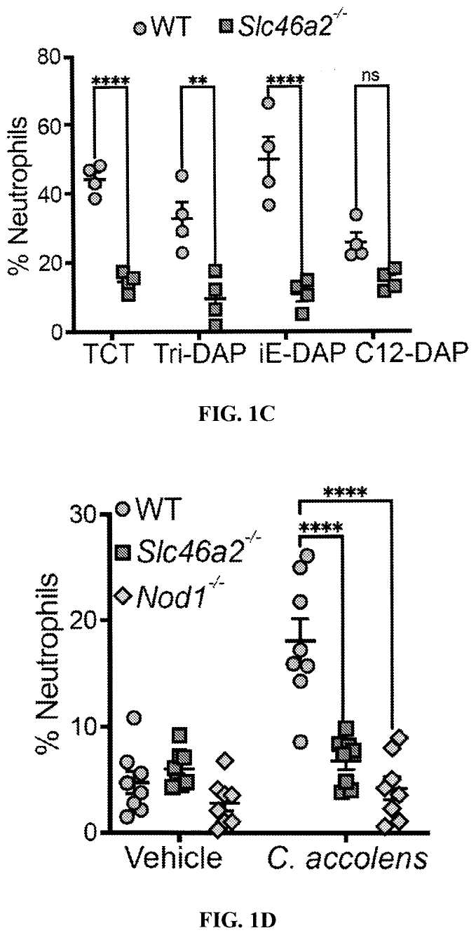

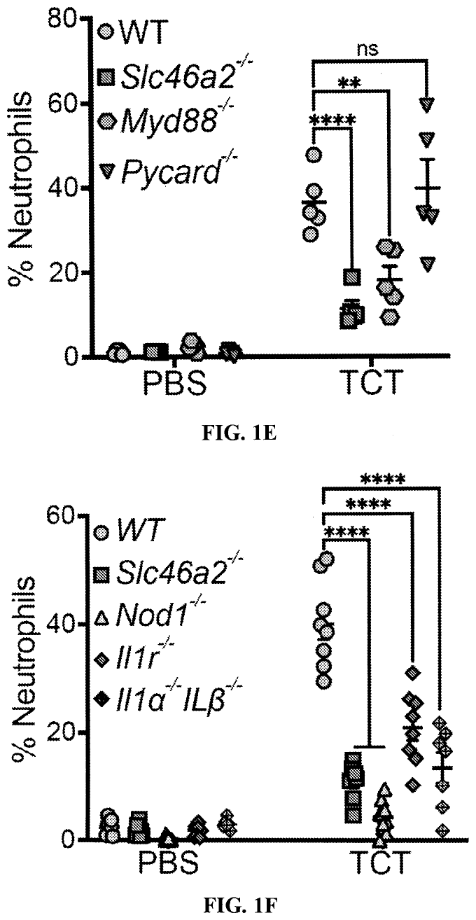

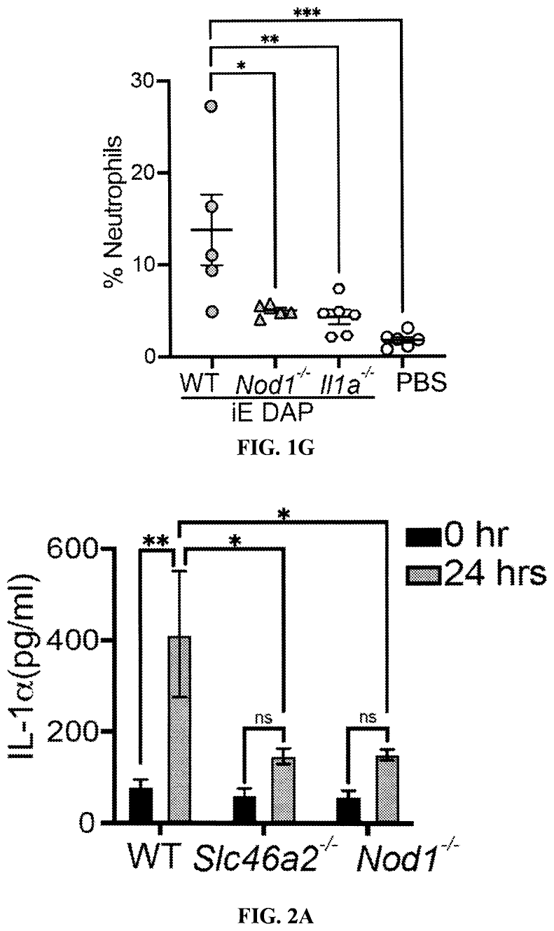

FIGS. 1A-1G: Slc46a2 is required for neutrophil recruitment in response to NOD1 stimulation in the mouse peritoneum and skin. FIGS. 1A-1B: Neutrophil recruitment after 3 h of intraperitoneal or intradermal injection of 10 μl of iE-DAP (30 μM) or MDP (10 μM). FIG. 1C: Neutrophil recruitment after 3 h intradermal injection of different DAP-type muropeptides, TCT (8 μM), iEDAP (30 μM), Tri-DAP (25 μM) or C12-iE-DAP (20 μM). FIG. 1D: Neutrophil recruitment after topical association of tape stripped skin with C. accolens. FIGS. 1E-1G: Recruitment of neutrophils after 3 h of intradermal injection of 10 μl of 8 μM TCT or 30 μM iE-DAP. Genotypes indicated on all panels. Comparisons two-way ANOVA with Tukey's multiple comparisons test to determine significance. ****P<0.0001; ***P<0.001; **P<0.01; *P<0.05; ns, not significant.

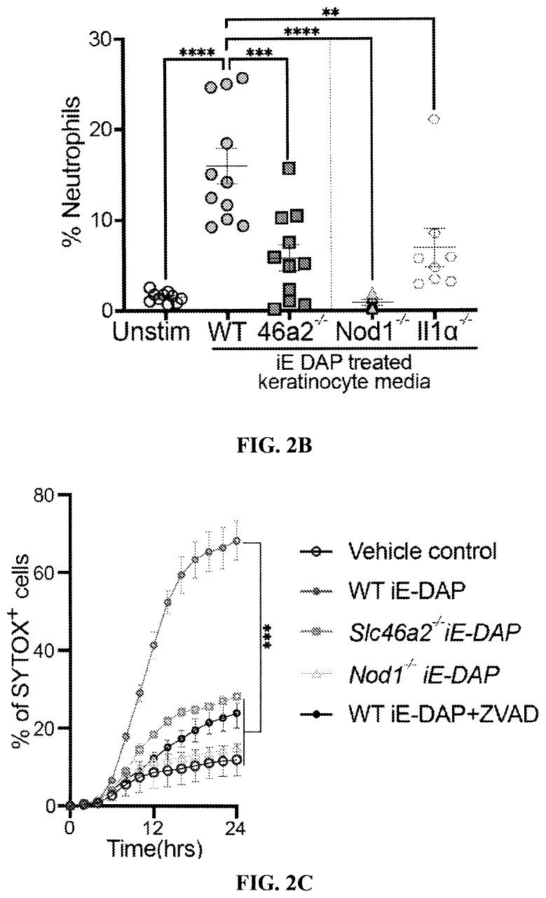

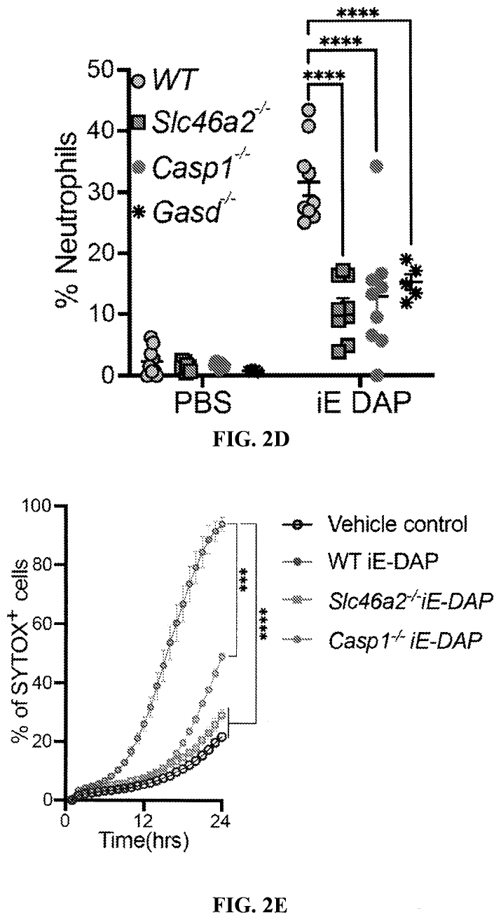

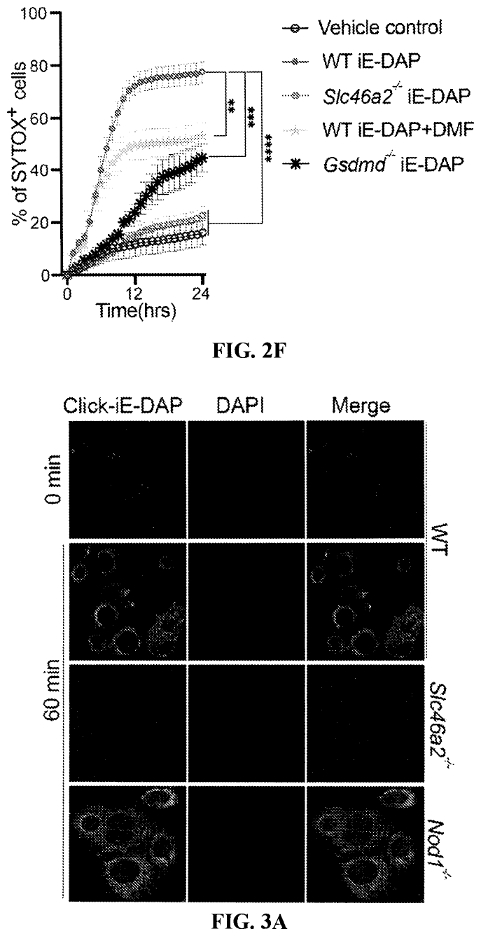

FIGS. 2A-2F: Primary mouse epidermal keratinocytes respond to DAP-muropeptides via Slc46a2 and Nod1. FIG. 2A: Keratinocytes released IL-1α following stimulation with 30 μM iE DAP for 24 h. FIG. 2B: Neutrophils recruitment to the peritoneum after IP injection of conditioned media from WT, Slc46a2^{-/-}, Nod1^{-/-} or Il1a^{-/-} keratinocytes stimulated with 30 μM iE-DAP for 24 h. FIG. 2C: Sytox dye uptake by keratinocytes by live cell imaging over 24 h following 30 μM iE-DAP stimulation. FIG. 2D: Neutrophil recruitment after 3 h intradermal injection of 10 μl of 30 μM iE-DAP. FIGS. 2E-2F: Sytox dye uptake by keratinocytes over 24 h following 30 μM iE-DAP challenge. Genotypes, caspase inhibitor zVAD-fmk (10 μM), or GasderminD inhibitor DMF (50 μM) indicated on all panels. FIGS. 2A-2B and FIG. 2D use two-way ANOVA and test, while FIG. 2C and FIGS. 2E-F use one-way ANOVA, and Tukey's multiple comparisons test to determine significance. ****P<0.0001; ***P<0.001; **P<0.01; *P<0.05; ns, not significant. n≥3 for all panels.

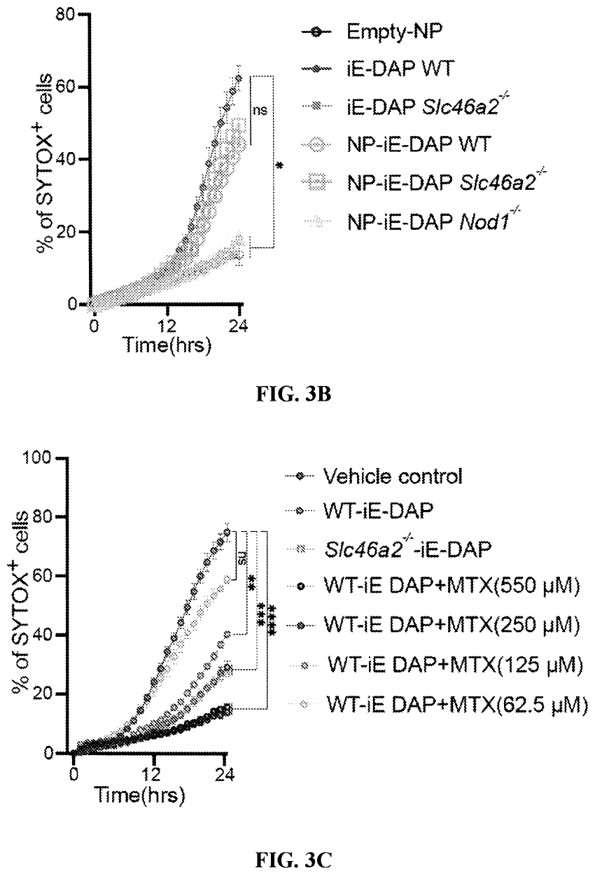

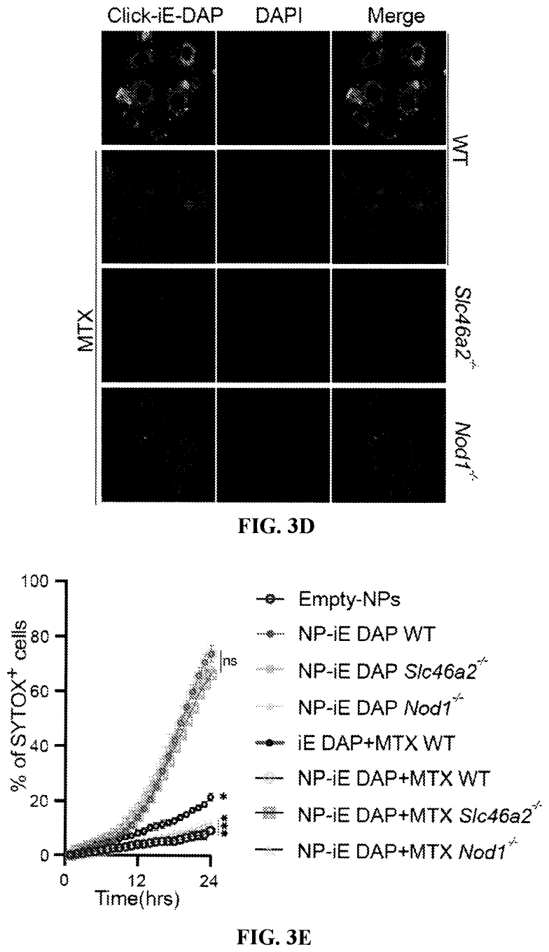

FIGS. 3A-3E: DAP-muropeptide transport requires Slc46a2 and is blocked by methotrexate. FIG. 3A and FIG. 3D: Confocal images of keratinocytes after 1 h challenge with 30 μM “click-iE-DAP” (FIG. 3A) or with 30 μM “click-iE DAP” and 250 μM methotrexate (MTX) (FIG. 3D), fixed, and then visualized with click reacted Calflour488-azide. FIGS. 3B-3C and FIG. 3E: Sytox dye uptake by keratinocytes over 24 h following stimulation with lipid nanoparticles (NP) loaded with iE-DAP (FIG. 3B), or treated increasing concentration of MTX and challenged with 30 μM iE-DAP (FIG. 3C), treated with 250 μM MTX and stimulated with iE-DAP-loaded NP. Genotypes indicated on all panels. FIGS. 3B-3C and FIG. 3E uses one-way ANOVA, and Tukey's multiple comparisons test to determine significance. ****P<0.0001; ***P<0.001; **P<0.01; *P<0.05; ns, not significant. n≥3 for all panels. FIG. 3A and FIG. 3D representative images from at least three independent experiments.

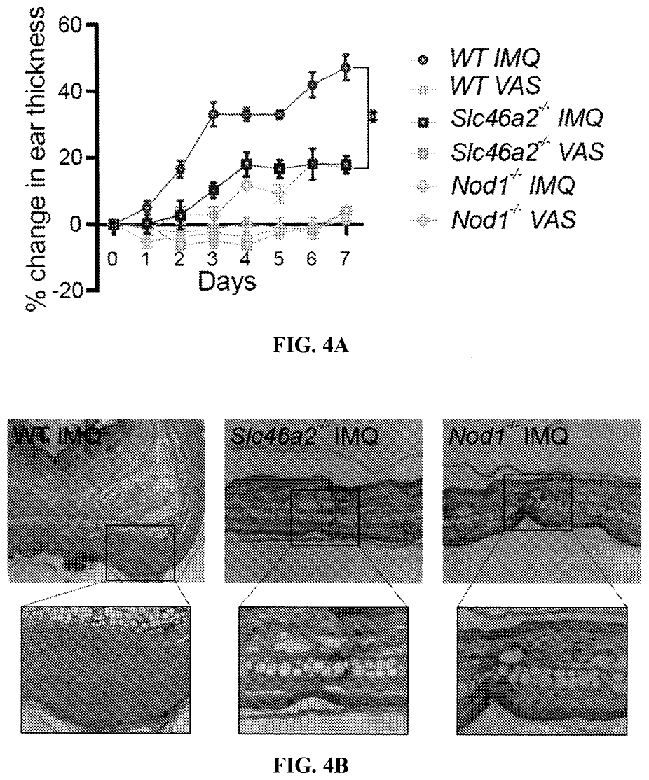

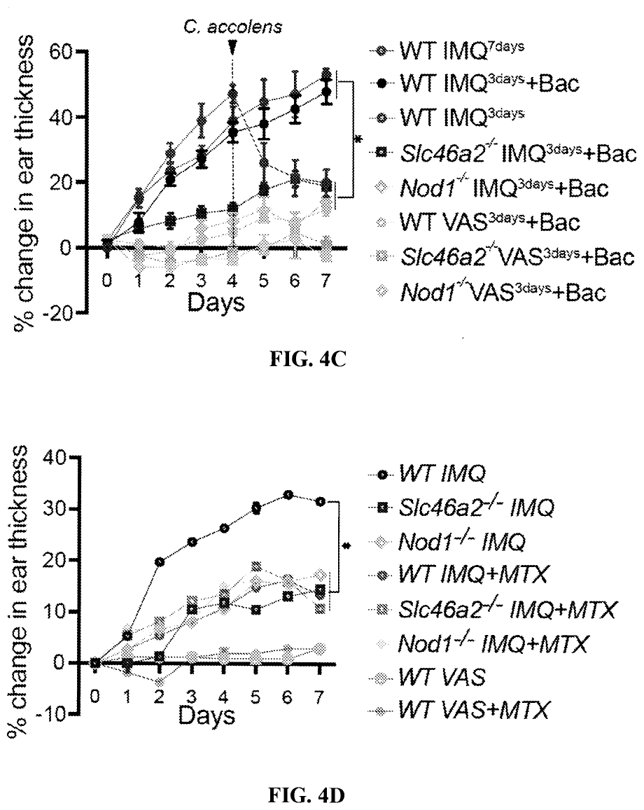

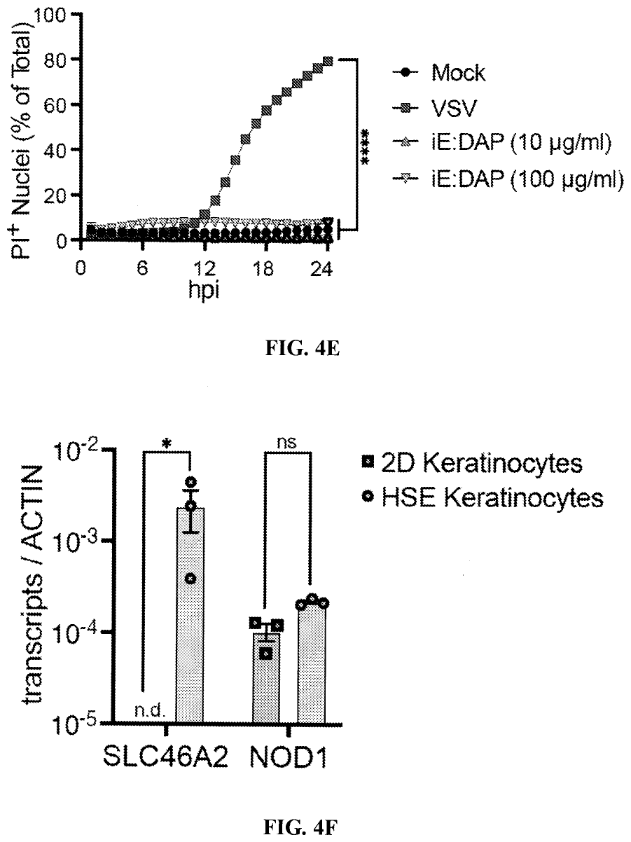

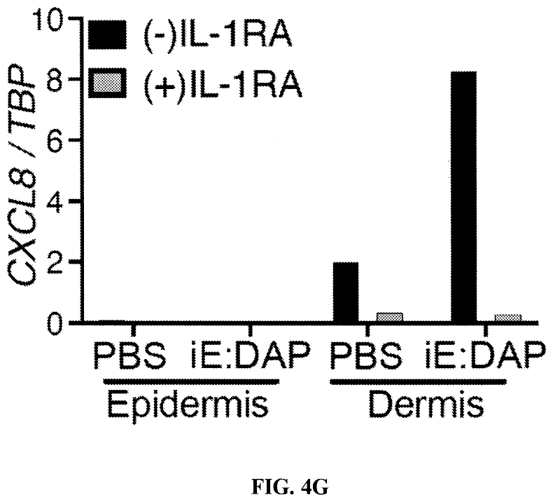

FIGS. 4A-4G: Slc46a2^{-/-} and Nod1^{-/-} mice are resistant to IMQ-induced psoriatic inflammation. FIG. 4A and FIGS. 4C-4D: 5% Imiquimod (IMQ) was topically applied to pinnae daily to induce psoriasis and ear inflammation was quantified daily. Contralateral pinnae were treated with Vaseline (VAS) as vehicle control. FIG. 4A: mice were treated with IMQ daily for 7 days and mean pinnae thickness is plotted. FIG. 4B: H&E stained histology images from Imiquimod treated pinnae on day 7. Genotypes are indicated on all panels. FIG. 4C: IMQ was applied for only 3 days, and then pinnae were treated daily for 3 days with topical application of live C. accolens (10⁷ CFU), except for controls with either a full 7 days or just 3 days of IMQ treatment. FIG. 4D: similar to FIG. 4A except IMQ was applied daily along with 50 μl volument of 250 μM MTX. FIG. 4E: Propidium Iodide (PI) uptake assay using primary human foreskin keratinocytes challenged with indicated iE-DAP doses or VSV virus infection (MOI 10) as a positive control. iE-DAP treatment did not induce cell permeabilization in human keratinocytes. FIG. 4F: Expression analysis of SLC46A2 and NOD1 in keratinocytes grown in 2D culture and 3D organoids (HSE). FIG. 4G: Induction of CXCL8 in skin organoid epidermal and dermal layers after PBS or iE-DAP challenge in the presence and absence of IL-1 receptor inhibitor (IL-1RA). High expression of CXCL8 was observed in dermis following iE-DAP challenge, and this was blocked by IL-IRA. FIG. 4A and FIGS. 4C-4E use oneway ANOVA and FIG. 4F uses two-way ANOVA test. Tukey's multiple comparisons test was used to determine significance. FIG. 4B and FIG. 4G are representative of three independent experimental results. ****P<0.0001; ***P<0.001; **P<0.01; *P<0.05; ns, not significant. n≥3 for all panels.

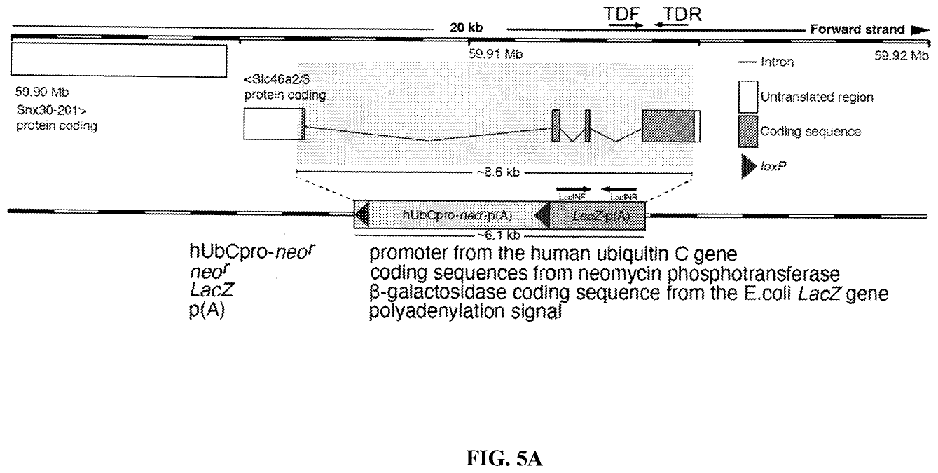

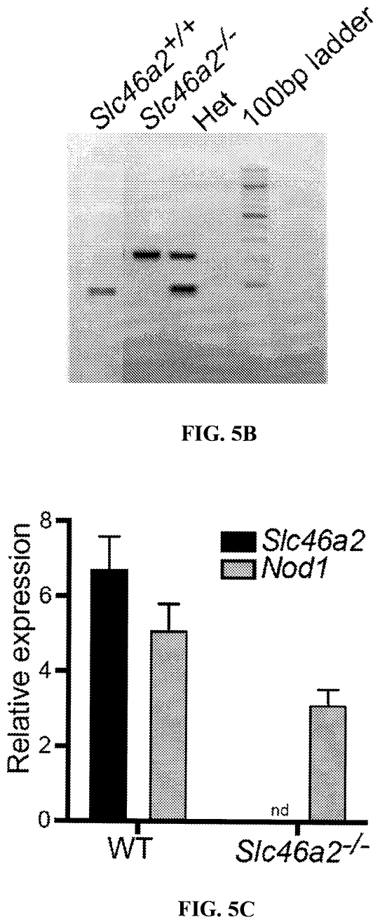

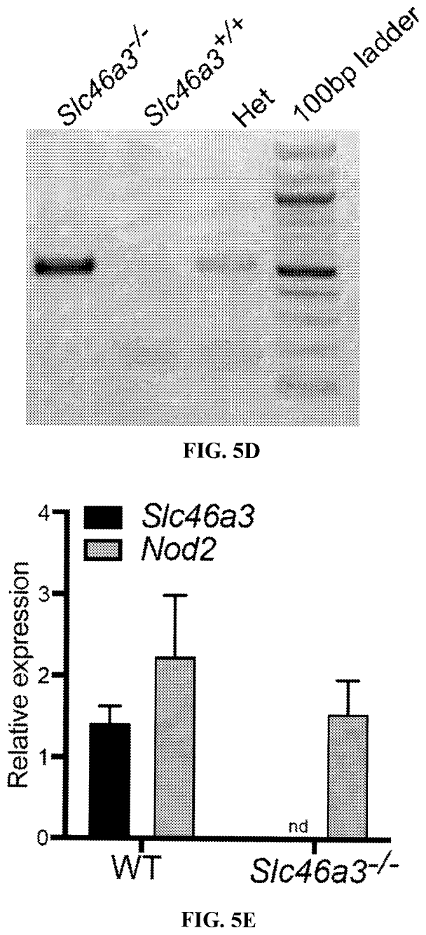

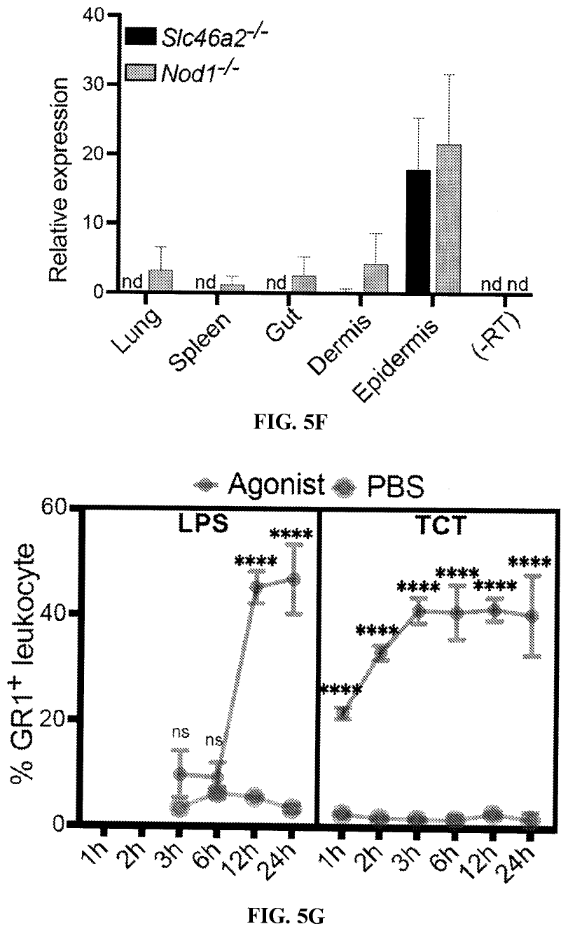

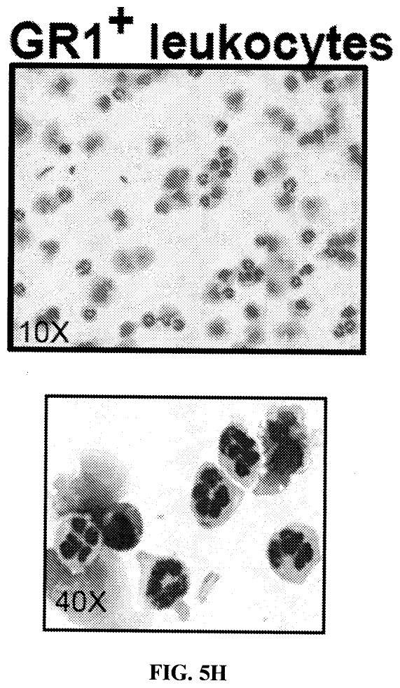

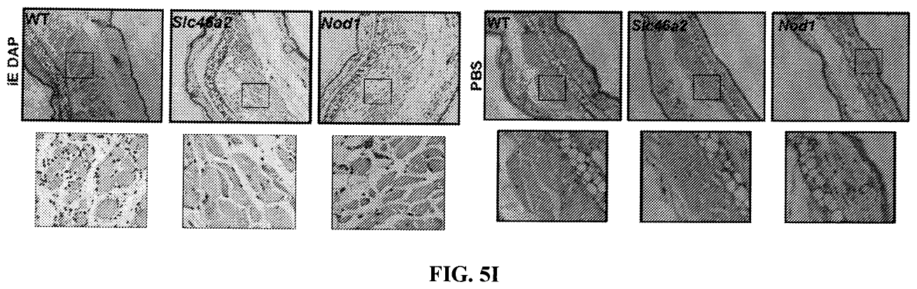

FIGS. 5A-5I: Slc46a2 Knockout Strategy and validation and recruitment of neutrophils in response to DAP muropeptide challenge. FIG. 5A: Design of Slc46a2/3 null allele. The gene region was replaced with ZEN-UB1 targeting cassette by homologous recombination. Primers used for genotyping are indicated. Adapted from velocigene (velocigene.com) and Ensembl genome browser. FIG. 5B: Agarose gel of PCR genotyping for validation of Slc46a2 knockout mice. FIG. 5C: Quantitative RT-PCR from WT and Slc46a2^{-/-} mouse epidermis for Slc46a2 and Nod1 expression. Slc46a2^{-/-} epidermis showed no Scl46a2 expression. FIG. 5D: Agarose gel of PCR genotyping for validation of Slc46a3 knockout mice. FIG. 5E: Quantitative RT-PCR from WT and Slc46a3^{-/-} mouse epidermis for Slc46a3 and Nod1 expression. Slc46a3^{-/-} epidermis showed no Slc46a3 expression. FIG. 5F: Expression analysis of Slc46a2 and Nod1 in indicated mouse organs. Maximum expression of both Slc46a2 and Nod1 was observed in epidermis. nd; not detected. FIG. 5G: Neutrophil recruitment to the pinnae was measured at indicated time points after intradermal injection of 10 μl of 10 μg/ml LPS or 8 μM TCT in one ear compared to a similar volume of PBS injection, as a control, in the contralateral ear. TCT triggered robust and rapid neutrophil recruitment in the skin while the response to LPS is slower. FIG. 5H: Images of FACS sorted GR1⁺ neutrophils 3 hr after iE-DAP challenge from WT mouse skin. Cells, prepared using cytospin and stained with Giemsa stain, show multilobed nuclei. FIG. 5I: Representative H&E stained histological sections from the mouse ear skin after 3 hr intradermal injection of 10 μl of 30 μM iE-DAP or equal volume of PBS in WT, Slc46a2^{-/-}, and Nod1^{-/-} mice. Inset shows the zoomed area of images. iE-DAP recruited inflammatory cells in WT but not in Slc46a2^{-/-}, and Nod1^{-/-} mice skin, whereas PBS injection did not induce inflammatory reaction. FIG. 5G uses two-way ANOVA and Tukey's multiple comparisons test to determine significance. FIGS. 5B-5F and FIGS. 5H-5I are representative of at least three independent experimental results. ****P<0.0001; **P<0.01; ns, not significant. n≥3 for all panels.

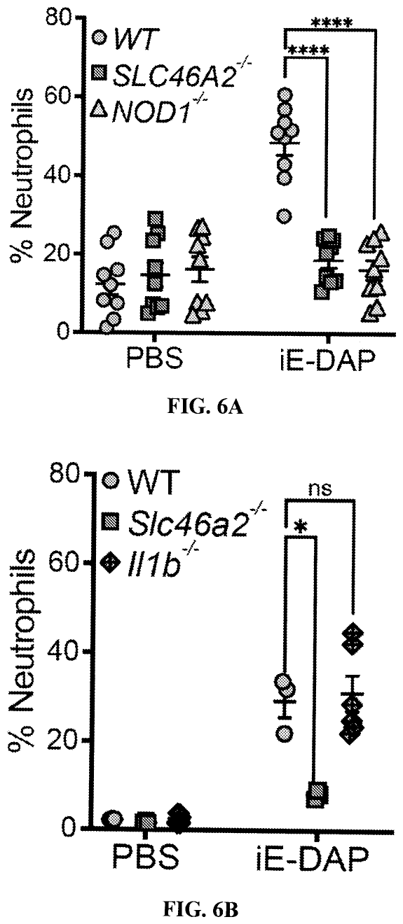

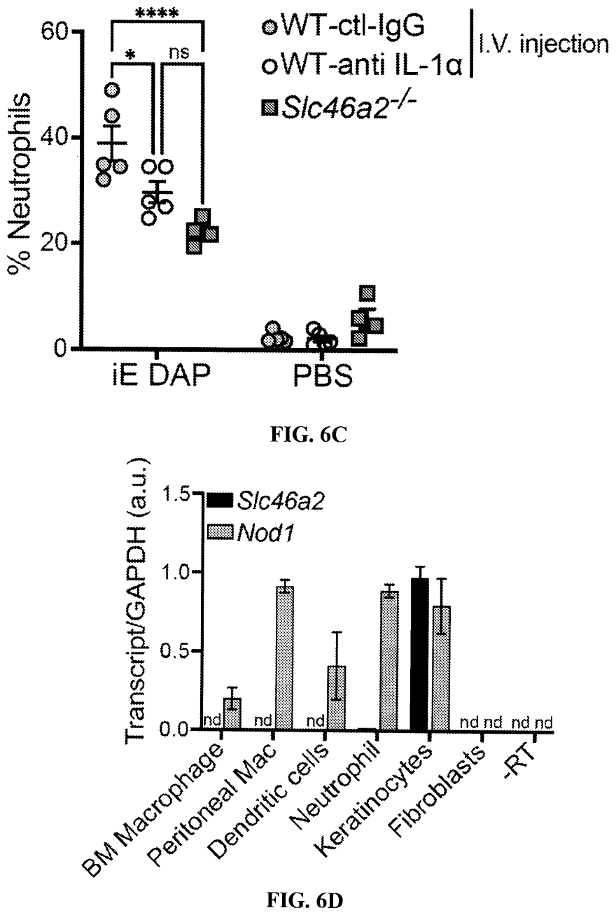

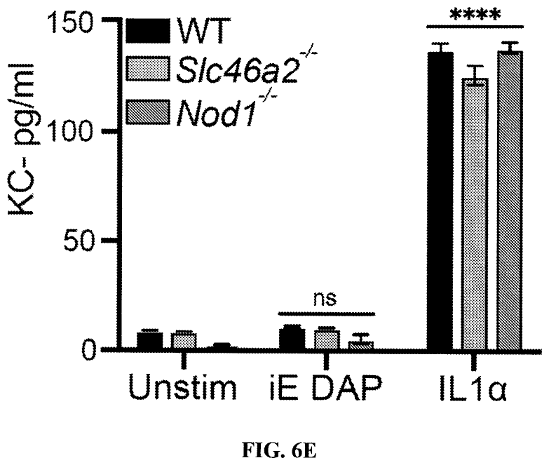

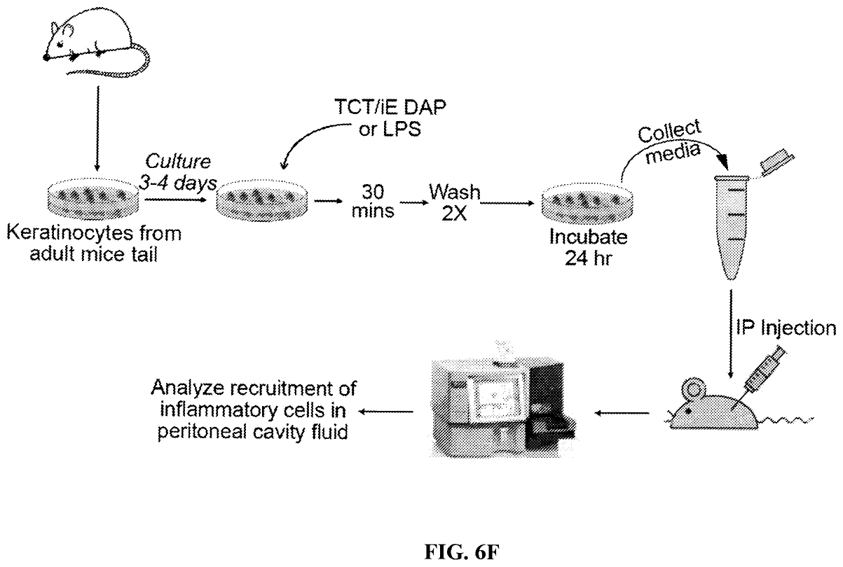

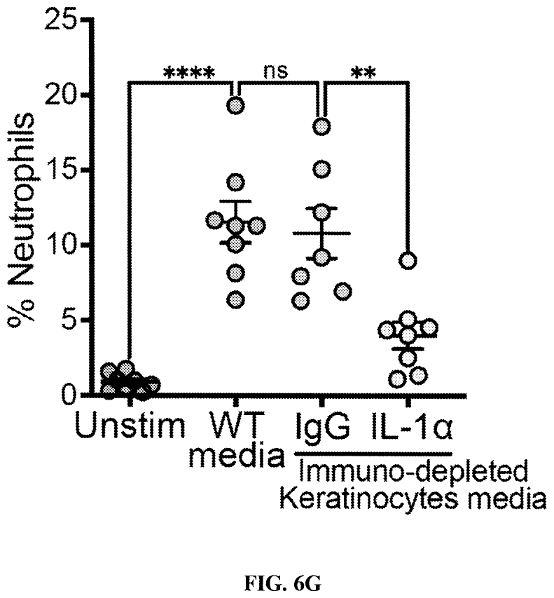

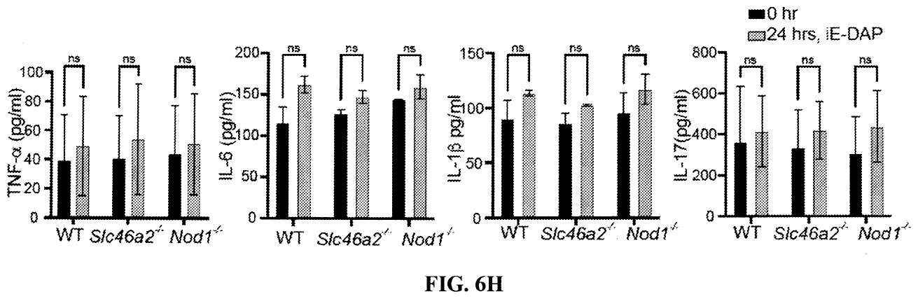

FIGS. 6A-6H: Slc46a2-dependent response to DAP-muropeptides recruits neutrophils and induces IL-1α. FIG. 6A: Neutrophil recruitment was measured in WT, Slc46a2^{-/-} or Nod1^{-/-} mice pinnae 3 h after topical application of 10 μl of 30 μM iE-DAP to tape stripped mouse skin. WT skin responded to iE-DAP challenge unlike Slc46a2^{-/-} and Nod1^{-/-} skin. FIG. 6B: Neutrophil recruitment was measured in WT, Il1b^{-/-} and Slc46a2^{-/-} mice pinnae in response of intradermal challenge with 10 μl of 30 μM iE-DAP. No significant difference was observed in GR1⁺ cell recruitment in IL1b^{-/-} mice compared to WT. FIG. 6C: Mice were intravenously injected with IL-1α blocking antibody (1 μg/mouse), or isotype matched IgG control, 1 hour prior to intradermal injection of 10 μl of 30 μM iE-DAP, and 3 h later neutrophil recruitment was measured. Neutralization of IL-1α significantly reduced the recruitment of leukocytes in WT mice. FIG. 6D: Expression analysis of Slc46a2 and Nod1 in specific cell types isolated from mice, by qRT-PCR. Highest expression of Slc46a2 was observed in keratinocytes. FIG. 6E: Primary dermal fibroblasts from WT, Slc46a2^{-/-} and Nod1^{-/-} mice were challenged with iE-DAP (30 μM) or IL-1α (10 ng/ml) for 24 hrs and CXCL1 (KC) was measured by ELISA from culture media. KC was induced in fibroblasts treated with IL-1α, regardless of their Slc46a2 or Nod1 genotype, but fibroblasts were unresponsive to iE-DAP. FIG. 6F: Schematic representation of experimental design for preparation of and bioassay analysis of conditioned media from primary mouse keratinocyte cultures. FIG. 6G: Anti-IL-1α antibody was used to deplete this cytokine from keratinocyte conditioned media prior to IP injection. IL-1α depleted media showed reduced neutrophil recruitment compared to control IgG antibody-treated media. FIG. 6H: TNFα, IL-6, IL-1β and IL-17 levels from WT, Slc46a2^{-/-} and Nod1^{-/-} keratinocyte media before and after challenge with 30 μM iE-DAP for 24 hrs. No significant induction in any of these cytokines was detected. FIGS. 6A-6C, FIG. 6E, and FIGS. 6G-6H use two-way ANOVA and Tukey's multiple comparisons test to determine significance. FIGS. 6D-6E and FIG. 6H are representative of at least three independent experimental results. ****P<0.0001; **P<0.01; ns, not significant. n≥3 for all panels.

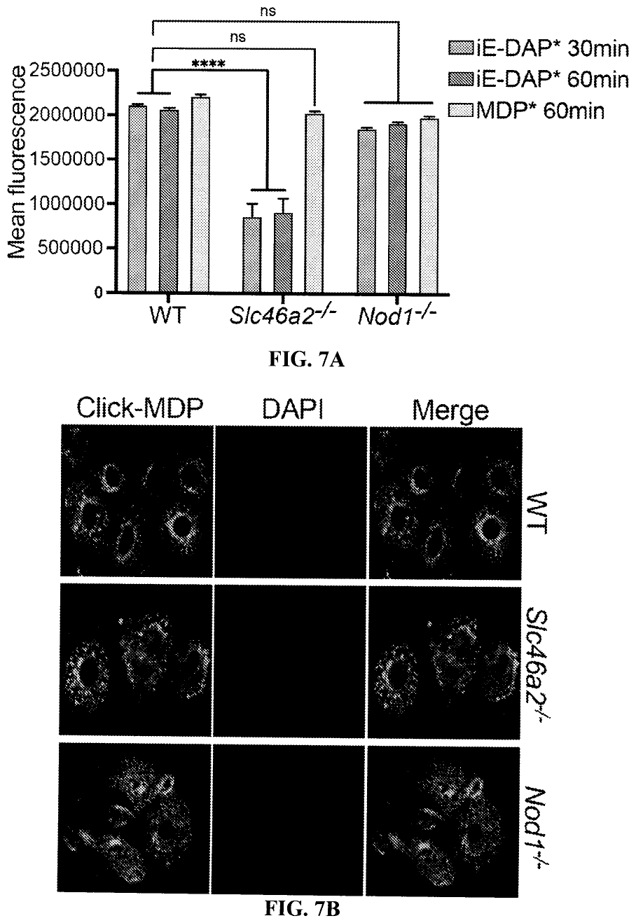

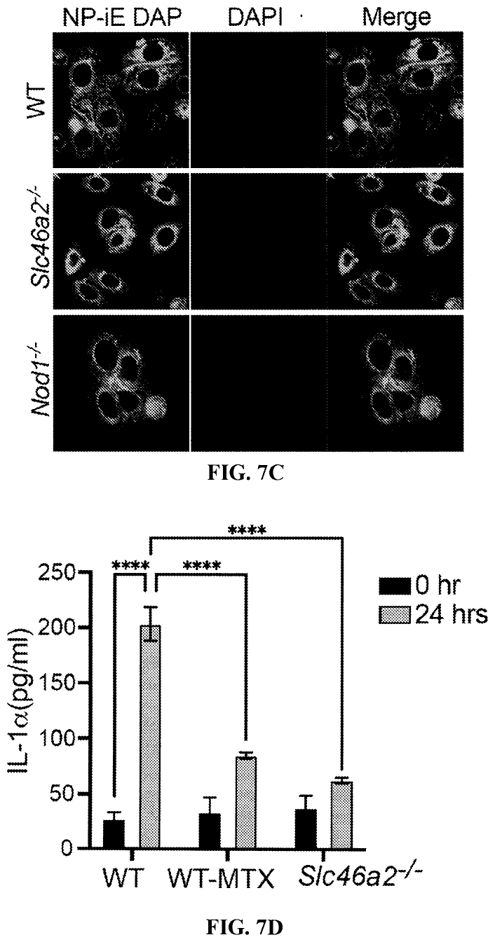

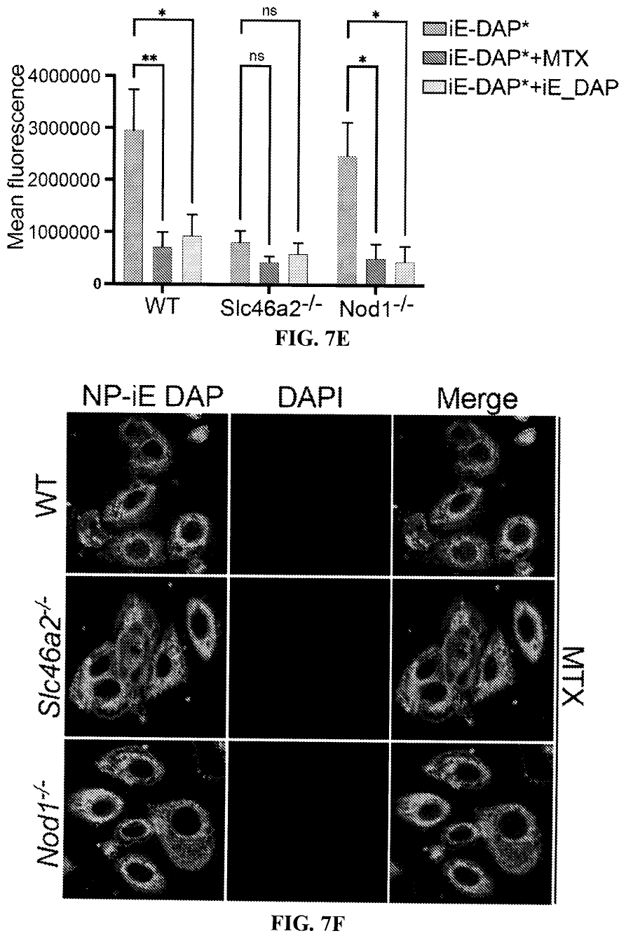

FIGS. 7A-7F: MTX blocks the transport of DAP muropeptides through SLC46A2. FIG. 7A: WT, Slc46a2^{-/-} and Nod1^{-/-}, keratinocytes were challenged with 30 μM click-iE DAP or 10 μM click-MDP for 30 minutes and 60 minutes, washed extensively, and then lysed. Muropeptides were then detected in these lysates with click-reacted CalFluor 488 Azide and fluorescence quantified. With iE-DAP, Slc46a2^{-/-} keratinocytes showed significantly reduced fluorescence intensity compared to WT or Nod1^{-/-} keratinocytes, while no change in fluorescence intensity was observed with click-MDP in all genotypes. FIG. 7B: Fluorescent confocal microscopy of WT, Slc46a2^{-/-} and Nod1^{-/-} keratinocytes, challenged with 10 μM “click-MDP” for 1 h. WT, Slc46a2^{-/-} and Nod1^{-/-} keratinocytes shows similar import of click-MDP. FIG. 7C: Fluorescent confocal microscopy of WT, Slc46a2^{-/-} and Nod1^{-/-} primary keratinocytes treated with NPs loaded with iE-DAP and immunofluorescence dye. Localization of dye inside the keratinocytes shows the successful delivery NP delivery of cargo. No change in fluorescence intensity was observed in all genotypes. FIG. 7D: IL-1α in culture media of WT and Slc46a2^{-/-} keratinocytes after stimulating with 30 μM iE DAP for 24 hours. WT keratinocytes were also treated with 250 μM MTX along with iE-DAP. Like Slc46a2-deficiency, MTX prevented IL-1α release in iE-DAP treated WT keratinocytes. FIG. 7E: Similar to FIG. 7A, MTX (250 μM) or unlabeled iE-DAP (30 μM) interfered with the cellular uptake of click-iE-DAP in WT or Nod1^{-/-}, keratinocytes, while in Slc46a2^{-/-} cells import was low and unchanged. FIG. 7F: Immunofluorescent images of WT, Slc46a2^{-/-} and Nod1^{-/-} keratinocytes pretreated with 250 μM MTX and then iE-DAP delivered by dye-loaded NP. Addition of MTX did not affect the NP mediated dye delivery in any genotype. FIG. 7A and FIGS. 7D-7E use two-way ANOVA and Tukey's multiple comparisons test to determine significance. FIGS. 7B-C and FIG. 7F are representative of at least three independent experimental results. ****P<0.0001; **P<0.01; ns, not significant. n≥3 for all panels.

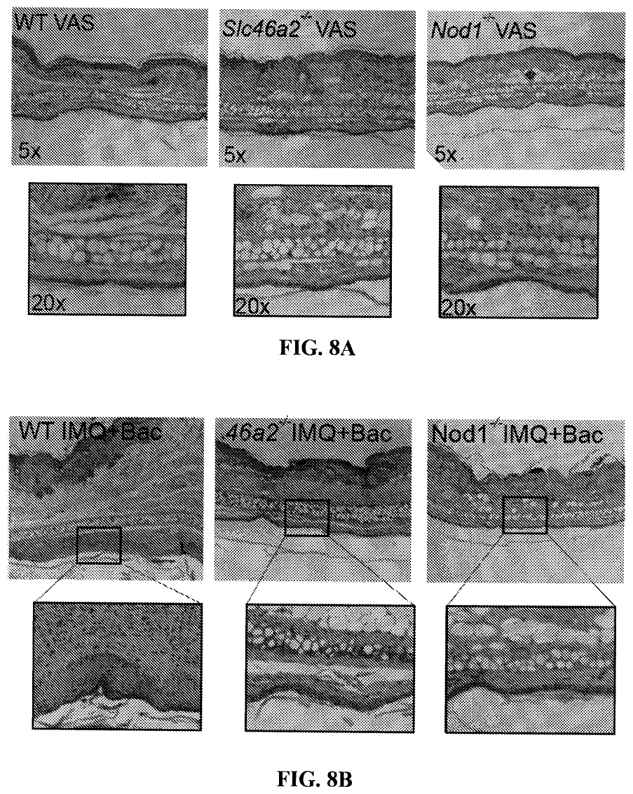

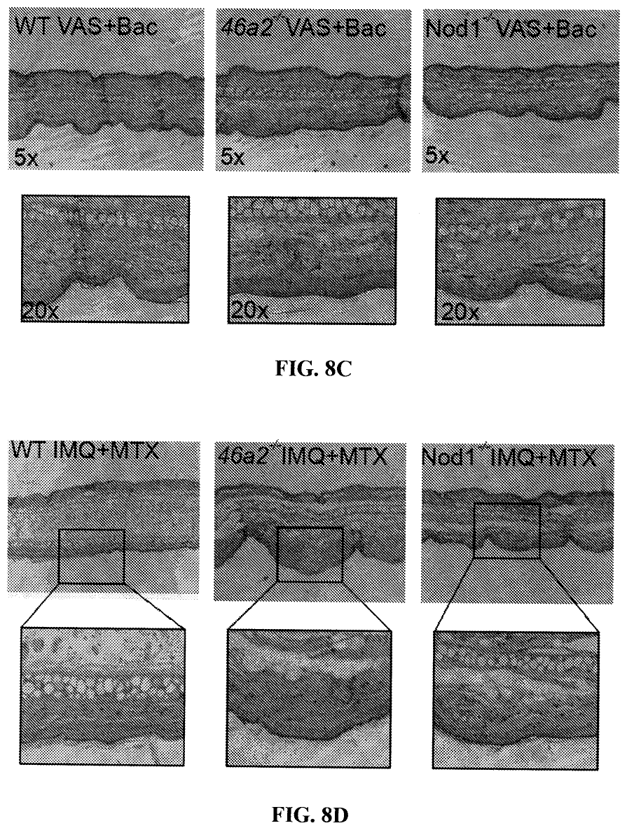

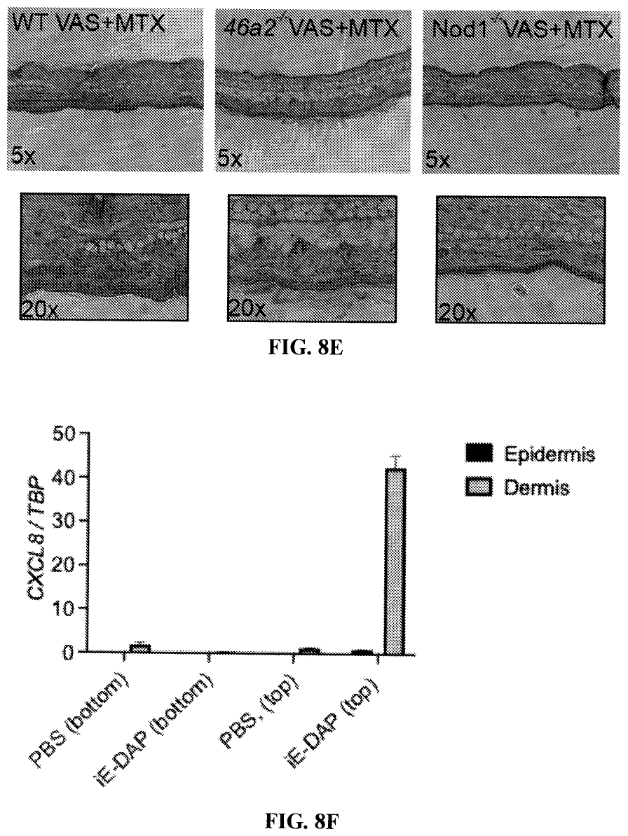

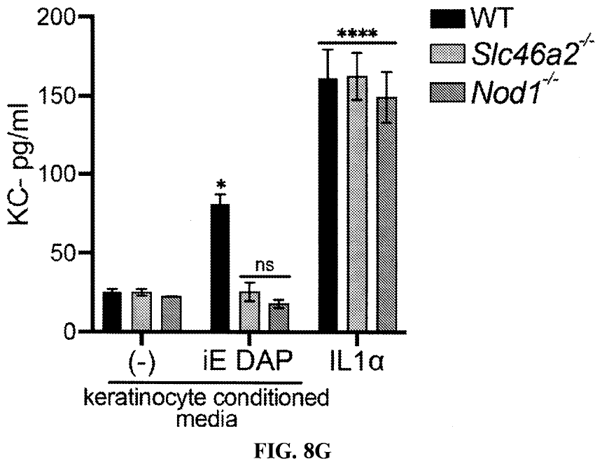

FIGS. 8A-8G: MTX blocks the psoriatic inflammation in skin. FIG. 8A: Representative H&E stained histology of ear sections from VAS (Vaseline, as vehicle) applied skin from WT, Slc46a2^{-/-} and Nod1^{-/-} mice. VAS applied skin did not show signs of inflammation in any genotype. FIG. 8B: H&E stained histology sections from IMQ and C. accolens (Bac) applied skin. WT skin shows hyper inflammation compared to Slc46a2−/− and Nod1−/− mice skin. FIG. 8C: Representative H&E stained histology of ear sections from VAS (Vaseline, as vehicle) and C. accolens (Bac) applied skin from WT, Slc46a2^{-/-} and Nod1^{-/-} mice. VAS applied skin did not show signs of inflammation in any genotype. FIG. 8D: H&E stained histology sections from IMQ and methotrexate (MTX) treated skin from from WT, Slc46a2^{-/-} and Nod1^{-/-} mice. After application of MTX WT skin was less inflamed. However, Slc46a2^{-/-} and Nod1^{-/-} skin has a minimalistic effect of MTX. FIG. 8E: Representative H&E stained histology of ear sections from VAS (Vaseline, as vehicle) and methotrexate (MTX) applied skin from WT, Slc46a2^{-/-} and Nod1^{-/-} mice. VAS applied skin did not show signs of inflammation in any genotype. FIG. 8F: CXCL8 expression was analyzed in skin organoids after iE-DAP challenge in the top (epidermal layer) or bottom (dermal layer). CXCL8 response was only observed in dermal fibroblast when an iE-DAP challenge was given to the epidermal keratinocytes. FIG. 8G: WT primary dermal fibroblasts were cultured for 24 h in conditioned media from WT, Slc46a2^{-/-} or Nod1^{-/-} keratinocytes, that were stimulated or not with 30 μM iE-DAP. As a control WT, Slc46a2^{-/-} and Nod1^{-/-} fibroblasts were treated with IL1-α (10 ng/ml). KC was induced in fibroblasts cultured in condition media from WT keratinocytes treated with iE-DAP or fibroblasts treated with IL1-α. FIG. 8G uses two-way ANOVA and Tukey's multiple comparisons test to determine significance. FIGS. 8A-8F are representative of at least three independent experimental results. ****P<0.0001; **P<0.01; ns, not significant. n≥3 for all panels.

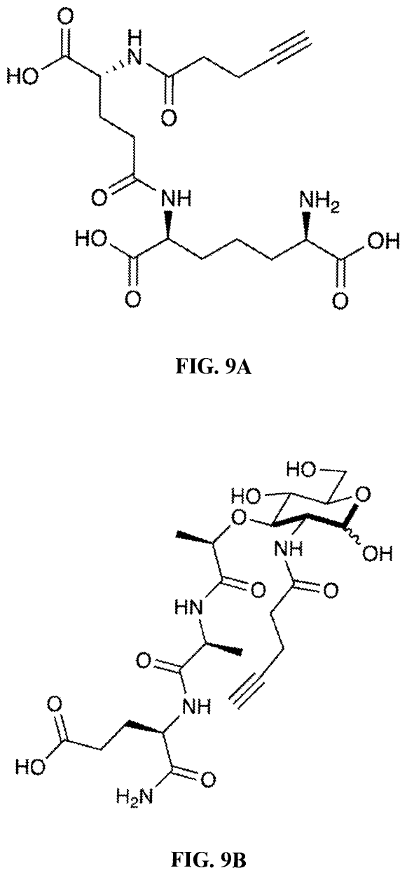

FIGS. 9A-9B: structure of biologically active click-iE-DAP (FIG. 9A) and click-MDP (FIG. 9B). The compound contains an alkyne on the amino-terminus of the dipeptide and the 2-amino group of the muramic acid sugar.

### DETAILED DESCRIPTION

The present disclosure is based, at least in part, on the unexpected finding that disruption of SLC46A2 reduces skin inflammation induced by imiquimod and diaminopimelic acid (DAP)-muropeptides in a mouse model of psoriasis. It was also demonstrated that disruption of SLC46A3 reduces skin inflammation induced by the muropeptide MDP.

Cytosolic innate immune receptors play critical roles in host defense by sensing microbial products that access the cell interior and activating potent immune and inflammatory responses (1, 2). These receptors include several nucleic acids sensors, such as cGAS, RIG-I, NLRP1 and AIM2, as well as others that respond to bacterial products (NOD1/2, NAIPs/NLRC4) or danger signals (NLRP1/3) (2-7). In some cell types, ligands for these cytosolic sensors can be imported into the cytosol (8-12). For instance, fragments of the bacterial cell wall, or muropeptides, enter the cytosol and drive potent inflammatory responses by activating NOD1/2 and NF-kB (13, 14). Muropeptides that contain the amino acid diaminopimelic acid (DAP), common to gram-negative bacteria and gram-positive bacilli, are potent NOD1 agonists, while muramyl-dipeptide (MDP), derived from nearly all bacterial peptidoglycan, activates NOD2.

While muropeptides can enter into some cell types, the underlying mechanisms of entry are unclear (15). Several reports have linked the solute carrier 15 (SLC15) family of peptide transporters to cytosolic muropeptide delivery (16-21). However, these solute carriers have not been directly demonstrated to transport muropeptides and are not specifically required for NOD signaling, but instead have recently been linked to IRF5 activation following stimulation of TLRs as well as NODs (22, 23). On the other hand, we recently identified the SLC46 family as candidate muropeptide transporters in Drosophila and mammalian cells (24). In particular, human or mouse SLC46A2 or SLC46A3, but not SLC46A1 (encoding the Proton Coupled Folate Transporter), strongly enhanced muropeptide-triggered NOD-dependent NF-kB reporter activity in cell lines. SLC46A2 is particularly intriguing as it was highly effective in delivering the DAP-containing muropeptide tracheal cytotoxin (TCT) to the cytosolic innate immune receptor NOD1 in these reporter assays, yet it is expressed in only a limited set of tissues. Analysis of publicly available databases and previous publications reveals that Slc46a2 is predominantly expressed in the skin epidermis as well as cortical epithelial cells of the thymus (25-27). Skin is a critical barrier defense against micro-organisms in the environment and an important immune-responsive organ (28, 29). NOD-mediated bacterial recognition plays a critical role in the interaction between gut microbiota and the intestinal epithelia (30), yet much less is known about the role of the NOD receptors in other barrier tissues, like the skin (31). Similar to the gut microbiome, the skin microbiome constantly interacts with the epidermis, modulating local and systemic immune responses, and is implicated in inflammatory skin diseases such as psoriasis (32-35). However, the role of NOD1/2 sensing in this tissue has not been evaluated.

Here we characterize a mouse deficient in Slc46a2, demonstrating its essential function for delivery of DAP-muropeptides and NOD1 activation in keratinocytes, and identifying a DAP-muropeptide triggered epidermal inflammatory response. This response involves Caspase-1 and Gasdermin D-dependent plasma membrane permeabilization of epidermal keratinocytes and the release of IL-1α. In the mouse, this response drives the rapid recruitment of neutrophils to muropeptide-challenged skin and is essential for the development of psoriatic inflammation. Moreover, we also show that this pathway is inhibited by the anti-folate methotrexate, indicating a novel mechanism of action for this commonly used anti-inflammatory drug (36). Human keratinocytes, in the context of 3D skin organoids, also similarly respond to DAP-muropeptides in an IL-1-dependent manner.

While it has been clear for many years that some cell types import muropeptides and activate the cytosolic innate immune receptors NOD1 or NOD2 (53), the underlying cellular and molecular mechanisms that mediate import of these molecules has remained unclear. Initially, the SLC15 family of oligopeptide transporters was implicated in this process (16-20), but they have never been demonstrated to bind or transport muropeptides while more recent studies instead have shown SLC15A4 functions as a scaffold involved in IRF5 activation downstream of TLR as well as NOD activation (20, 22). On the other hand, our initial work in Drosophila implicated SLC46s in this process (24), and here we show that SLC46A2 has the properties of a transporter selective for delivering DAP-type muropeptides for NOD1 activation in murine tissues. Using in vivo approaches in two different tissues, as well as ex vivo studies with primary mouse keratinocytes, Slc46a2 was uniquely required for the response to multiple NOD1 activating DAP-muropeptides, while Slc46a3 was required for the response to the NOD2 activating muropeptide MDP. Moreover, two orthogonal approaches were used to demonstrate that intracellular delivery of DAP-muropeptide requires Slc46a2. Intracellular iE-DAP, directly detected via click chemistry dye labeling, required Slc46a2 but not Nod1. On the other hand, when packaged in a lipo-nanoparticle for direct intracellular delivery, iE-DAP activation of NOD1 no longer required Slc46a2. Together, these immunological and cell biological data strongly argue for the direct transport of DAP-muropeptides by SLC46A2, and implicate SLC46A3 in the delivery of MDP, and not vice versa. Interestingly, primary human keratinocytes, in standard tissue culture conditions, do not express SLC46A2 and do not respond to iE-DAP, while after differentiating into a squamous epithelium in the HSE organoids, SLC46A2 is expressed and the epidermis responds to DAP-muropeptide.

In both humans and mice, Slc46a2 is expressed in only a limited set of tissues, including the epidermis (40, 41). This expression pattern led us to explore, in more detail, the role of SLC46A2/NOD1 dependent responses in skin. We found a robust neutrophilic response to intradermal DAP-muropeptide challenge that required Slc46a2 and Nod1. Similarly, we also found that murine skin, once the outer waxy stratum corneum was removed, responded to topical application of either iE-DAP or the DAP-producing skin commensal Corynebacterium accolens through Slc46a2 and Nod1. Likewise, primary mouse keratinocytes ex vivo responded to DAP-muropeptides with cell permeabilization and the release of active IL-1α. Similar to the in vivo neutrophilic response, the cell permeabilization pathway in keratinocytes involved Casp1 and Gsdmd in addition to Slc46a2 and Nod1. Together these results demonstrate that skin keratinocytes respond to DAP-muropeptides via intracellular delivery by SL46A2 and activation of NOD1, which in turn drives IL-1α release via pathway that involves Caspase-1 and Gasdermin D cell permeabilization, and perhaps cell death. The rapidity of the response in vivo suggests this is not a transcription-dependent response, although the response is slower in isolated keratinocytes.

Given the established link between C. accolens and psoriasis, in humans and mouse models (33-35), the role of this response in psoriatic inflammation was also examined in a mice model. Animals deficient for either Slc46a2 or Nod1 were strikingly resistant to psoriatic inflammation and unresponsive to C. accolens triggered pathology. Given the previous work linking C. accolens to the activation of IL-17 producing γδT cells in the skin (32), it will be interesting to learn if SLC46A2/NOD1 pathway in the skin also drives this IL-17 producing gd response, in addition to the strong neutrophilic infiltration.

Immune-mediated inflammatory diseases, like psoriasis, are often treated with the first line drug methotrexate. MTX is a complicated drug, originally developed as an antiproliferative due to its interference with folate-dependent enzymes required for nucleotide biosynthesis and DNA replication, that is still used as a cancer therapy. Subsequently, MTX was found to be effective in psoriasis, rheumatoid arthritis and other immune mediate inflammatory diseases, but at much lower doses that do not impact cell proliferation (36, 54). The mechanisms of action of MTX as an anti-inflammatory are controversial. The leading model argues that MTX functions as a traditional anti-folate interfering with the enzyme AICAR transformylase, leading to increased intracellular AICAR and eventually increased extracellular Adenosine, a known anti-inflammatory molecule that acts via adenosine receptors, such as P1-A2A (55). In addition, MTX has been argued to interfere with NF-kB activation through unknown mechanisms. Here we present a new mechanism of action for anti-inflammatory activity of MTX that does not involve interfering with folate-dependent enzymes. Instead, the data here shows that MTX competes with DAP-muropeptide for delivery into the cytosol, phenocopying Slc46a2-deficiency in three separate ex vivo keratinocytes assays as well as with a in vivo model of psoriatic inflammation. In vivo, MTX application in the Slc46a2 and Nod1 mutants had no effect on the residual inflammation still observed in the psoriasis model, while it strongly suppressed psoriatic-like inflammation in WT mice, to levels very similar to that observed in the mutants. Together, these in vitro and in vivo findings argue that MTX competes with DAP-muropeptides as cargo for the transporter SLC46A2, and thereby directly interferes with a central inflammatory pathway, the NOD1 pathway, which operates in the skin to drive neutrophil recruitment and psoriatic-like inflammation.

Through a combination of immunology, chemical biology and biomedical engineering approaches, we demonstrate that SLC46A2 functions to deliver DAP-muropeptides to the cytosol of keratinocytes, driving a robust IL-1α dependent inflammatory response and neutrophil recruitment. This response is critical for psoriatic-like inflammation in a mouse model. Additionally, the common anti-inflammatory drug MTX phenocopies Slc46a2 deficiency in vivo and in primary mouse keratinocytes, arguing that SLC46A2 is a direct anti-inflammatory target of this drug. Moreover, these findings identify disease mechanisms and therapeutic targets that could be applicable to a broad number of inflammatory skin conditions and highlight a novel role for the SLC46 family in host-microbiome interactions.

Accordingly, the present disclosure provides, in some aspects, therapeutic uses of inhibitors of SLC46A2 and/or inhibitors of SLC46A3 for treating inflammatory skin diseases such as psoriasis (e.g., plaque psoriasis, pustular psoriasis, guttate psoriasis, inverse psoriasis), skin infections, dermatitis, acne, rosacea, cutaneous lupus erythematosus (e.g., acute lupus erythematosus, subacute lupus erythematosus, chronic lupus erythematosus), lichen planus, cutaneous dermatomyositis, pityriasis rubra pilaris, and hidradenitis suppurativa. Non-limiting examples of dermatitis include atopic dermatitis (also known as eczema), allergic contact dermatitis, irritant contact dermatitis, nummular dermatitis, dyshidrotic dermatitis, stasis dermatitis, and seborrheic dermatitis.

### I. Inhibitors of SLC46A2 and/or Inhibitors of SLC46A3 and Pharmaceutical Compositions Comprising Such

The SLC46 transporter family has 3 paralogs in mice and humans. SLC46A1 is a proton-coupled folate transporter that is responsible for the intestinal absorption of folate and antifolates. SLC46A2 was first identified because of its abundant expression in mouse thymic cortical epithelial cells, but has not been functionally characterized. Likewise, SLC46A3 is not yet characterized. Exemplary amino acid sequences of human SLC46A1, SLC46A2, and SLC46A3 are provided in UniProt Identifier Q96NT5, Q9BY10, and Q7Z3Q1, respectively. Methods provided herein involve treating an inflammatory skin disease using an inhibitor of SLC46A2 and/or inhibitor of SLC46A3.

### (a) Inhibitors of SLC46A2 and/or Inhibitors of SLC46A3

The term “inhibitor,” as used herein, refers to a molecule (e.g., a small molecule or a biological molecule) that blocks, inhibits, reduces (including significantly), or interferes with SLC46A2 (e.g., mammalian SLC46A2 such as human SLC46A2) and/or SLC46A3 (e.g., mammalian SLC46A3 such as human SLC46A3) biological activity in vitro, in situ, and/or in vivo.

The term “inhibitor” implies no specific mechanism of biological action whatsoever, and expressly includes and encompasses all possible pharmacological, physiological, and biochemical interactions with SLC46A2 and/or SLC46A3 whether direct or indirect, and whether interacting with SLC46A2 and/or SLC46A3 or their substrates, or through another mechanism, and its consequences which can be achieved by a variety of different, and chemically divergent compositions.

In some examples, an inhibitor can be a molecule that inhibits or disrupts SLC46A2 itself (e.g., human SLC46A2), a biological activity of SLC46A2 (e.g., including but not limited to its ability to mediate any aspect of inflammation), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts SLC46A3 itself (e.g., human SLC46A3), a biological activity of SLC46A3 (e.g., including but not limited to its ability to mediate any aspect of inflammation), or the consequences of the biological activity to any meaningful degree, e.g., at least 10%, at least 20%, at least 30%, at least 40%, at least 50%, at least 60%, at least 70%, at least 80%, at least 90%, or more.

In some examples, an inhibitor can be a molecule that inhibits or disrupts SLC46A2 and SLC46A3, a biological activity of SLC46A2 and SLC46A3 (e.g., including but not limited to its ability to mediate any aspect of inflammation), or the consequences of the biological activity to any meaningful degree, e.g., at least 10%, at least 20%, at least 30%, at least 40%, at least 50%, at least 60%, at least 70%, at least 80%, at least 90%, or more.

Non-limiting examples of an inhibitor of SLC46A2 for use in the methods described herein include a small molecule, an agent that inhibits expression of SLC46A2 (e.g., a nucleic acid molecule that inhibit SLC46A2 expression such as a short interfering RNA (siRNA)), anti-SLC46A2 antibody, or a peptide that inhibits SLC46A2 (e.g., a peptide aptamer, a SLC46A2 structural analog). In some embodiments, the SLC46A2 inhibitor binds SLC46A2 (i.e., physically interacts with SLC46A2), binds to a binding partner of SLC46A2, and/or inhibits expression (i.e., transcription or translation) or processing of SLC46A2.

Non-limiting examples of an inhibitor of SLC46A3 for use in the methods described herein include a small molecule, an agent that inhibits expression of SLC46A3 (e.g., a nucleic acid molecule that inhibit SLC46A3 expression such as a short interfering RNA (siRNA)), anti-SLC46A3 antibody, or a peptide that inhibits SLC46A3 (e.g., a peptide aptamer, a SLC46A3 structural analog). In some embodiments, the SLC46A3 inhibitor binds SLC46A3 (i.e., physically interacts with SLC46A3), binds to a binding partner of SLC46A3, and/or inhibits expression (i.e., transcription or translation) or processing of SLC46A3.

Any small molecule suitable for inhibiting SLC46A2 and/or SLC46A3 can be used in methods described herein. The term “small molecule inhibitor of SLC46A2 and/or SLC46A3” refers to small organic compounds, inorganic compounds, or any combination thereof that inhibits or reduces SLC46A2 and/or SLC46A3 biological activity. In some embodiments, a small molecule inhibitor of SLC46A2 and/or SLC46A3 used in the methods described herein inhibits SLC46A2 and/or SLC46A3 biological activity by at least 10% (e.g., at least 20%, at least 30%, at least 40%, at least 50%, at least 60%, at least 70%, at least 80%, at least 90%, or more). Small molecule inhibitors can be used in methods described herein in the free form, as a salt thereof, a prodrug derivative thereof, or combinations thereof.

Any agent suitable for inhibiting expression of SLC46A2 and/or SLC46A3 can be used in methods described herein. In some embodiments, an agent that inhibits expression of SLC46A2 and/or SLC46A3 used in the methods described herein inhibits SLC46A2 and/or SLC46A3 expression by at least 10% (e.g., at least 20%, at least 30%, at least 40%, at least 50%, at least 60%, at least 70%, at least 80%, at least 90%, or more). In some examples, agents that inhibit expression of SLC46A2 and/or SLC46A3 are nucleic acid molecules such as short interfering nucleic acid (siNA), short interfering RNA (siRNA), double-stranded RNA (dsRNA), micro-RNA (miRNA), and short hairpin RNA (shRNA) molecules. Such nucleic acid molecules can include non-naturally-occurring nucleobases (e.g., modified nucleobases), sugars (e.g., substituted sugar moieties), and/or covalent internucleoside linkages (e.g., modified backbones). An exemplary nucleic acid sequence of human SLC46A2 and human SLC46A3 are provided in NCBI Reference Sequence NM_033051.3 and NM_001135919.2, respectively. Nucleic acids for inhibiting expression of SLC46A2 and SLC46A3 are commercially available, e.g., from Abnova, GeneCopoeia, and Santa Cruz Biotechnology.

In some examples, the inhibitor of SLC46A2 can be one or more molecules that disrupt the SLC46A2 gene. Any molecule(s) known in the art can be used to disrupt the SLC46A2 gene and/or the SLC46A3 gene including gene editing molecules such as guide RNA (gRNA) and clustered regularly interspaced short palindromic repeat (CRISPR)-associated 9 (Cas9) nuclease.

In some examples, the inhibitor of SLC46A2 to be used in methods described herein can be an anti-SLC46A2 antibody and/or an anti-SLC46A3 antibody. An anti-SLC46A2 antibody is an antibody capable of binding to SLC46A2, which can inhibit SLC46A2 biological activity and/or downstream pathway(s) mediated by SLC46A2 signaling. In some embodiments, an anti-SLC46A2 antibody used in the methods described herein inhibits SLC46A2 biological activity and/or downstream pathway(s) mediated by SLC46A2 signaling by at least 10% (e.g., at least 20%, at least 30%, at least 40%, at least 50%, at least 60%, at least 70%, at least 80%, at least 90%, or more). An anti-SLC46A3 antibody is an antibody capable of binding to SLC46A3, which can inhibit SLC46A3 biological activity and/or downstream pathway(s) mediated by SLC46A3 signaling. In some embodiments, an anti-SLC46A3 antibody used in the methods described herein inhibits SLC46A3 biological activity and/or downstream pathway(s) mediated by SLC46A3 signaling by at least 10% (e.g., at least 20%, at least 30%, at least 40%, at least 50%, at least 60%, at least 70%, at least 80%, at least 90%, or more). Antibodies that bind to SLC46A2 or to SLC36A3 are commercially available, e.g., from Abnova and Invitrogen.

An antibody is an immunoglobulin molecule capable of specific binding to a target, such as carbohydrate, polynucleotide, lipid, polynucleotide, lipid, polypeptide, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof such as Fab, Fab′, F(ab′)₂, Fv, single chain (scFv), mutants thereof, fusion proteins comprising antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An anti-SLC46A2 antibody or an anti-SLC46A3 antibody can be an antibody of any class, such as IgD, IgE, IgG, IgA, or IgM (or sub-class thereof), or the anti-SLC46A2 antibody or the anti-SLC46A3 need not be of any particular class.

In some examples, the inhibitor of SLC46A2 or the inhibitor of SLC46A3 to be used in methods described herein can be a peptide inhibitor. For example, the inhibitor of SLC46A2 can be a peptide comprising a portion of a SLC46A2-binding protein that specifically binds to SLC46A2 and blocks its biological activity and/or interaction with NOD1, a muropeptide, a SLC46A2 binding protein, or a combination thereof. For example, the inhibitor of SLC46A3 can be a peptide comprising a portion of a SLC46A3-binding protein that specifically binds to SLC46A3 and blocks its biological activity and/or interaction with NOD2, a muropeptide, a SLC46A3 binding protein, or a combination thereof.

In some embodiments, a peptide inhibitor used in the methods described herein inhibits SLC46A2 and/or SLC46A3 biological activity and/or downstream pathway(s) mediated by SLC46A2 and/or SLC46A3 signaling by at least 10% (e.g., at least 20%, at least 30%, at least 40%, at least 50%, at least 60%, at least 70%, at least 80%, at least 90%, or more).

Any of the polynucleotides and polypeptides described herein for inhibiting SLC46A2 and/or SLC46A3 can be included in a delivery vehicle. The delivery vehicle can be of viral (e.g., viral vectors) or non-viral origin (e.g., eukaryotic cell delivery vehicles). Viral vectors are known in the art and include, but are not limited to, retroviral vectors, lentiviral vectors, adenoviral vectors, adeno-associated viral vectors, herpes simplex viral vectors, and vaccinia viral vectors. Expression of coding sequences in the delivery vehicle can be induced using endogenous or heterologous promoters and/or enhances. Expression of the coding sequence can be either constitutive or regulated.

### (b) Pharmaceutical Compositions

Any inhibitor (e.g., those described herein) can be mixed with a pharmaceutically acceptable excipient (carrier) to form a pharmaceutical composition for use in treating an inflammatory skin disease (e.g., atopic dermatitis, psoriasis, bacterial infection). “Acceptable” means that the excipient must be compatible with the inhibitor (and preferably, capable of stabilizing the inhibitor) and not deleterious to the subject to be treated. Pharmaceutically acceptable excipients (carriers), including buffers, are well known in the art. See, e.g., Remington: The Science and Practice of Pharmacy 20^{th} Ed. (2000) Lippincott Williams and Wilkins, Ed. K. E. Hoover.

Pharmaceutical compositions comprising an inhibitor to be used in the methods described herein can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers (e.g., phosphate, citrate, and other organic acids); antioxidants (e.g., ascorbic acid, methionine); preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins (e.g., serum albumin, gelatin, immunoglobulins); hydrophilic polymers (e.g., polyvinylpyrrolidone); amino acids (e.g., glycine, glutamine, asparagine, histidine, arginine, lysine); monosaccharides, disaccharides, and other carbohydrates including glucose, mannose, or dextrans; chelating agents (e.g., ETDA) sugars (e.g., sucrose, mannitol, sorbitol); salt-forming counter-ions (e.g., sodium); metal complexes (e.g., Zn-protein complexes); and/or non-ionic surfactants (e.g., TWEEN™, PLURONICS™, polyethylene glycol (PEG)).

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the inhibitor with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used. Alternatively, tissue-coating solutions, such as pectin containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the inhibitor in the proper medium. Absorption enhancers can also be used to increase the flux of the inhibitor across the skin. The rate can be controlled by either providing a rate controlling membrane or by dispersing the agent in a polymer matrix or gel.

Additionally, the carrier for a topical formulation can be in the form of a hydroalcoholic system (e.g., gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids, and aqueous based mousse systems.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For preparing solid compositions such as tablets, the principal active ingredient (i.e., an inhibitor of SLC46A2, an inhibitor of SLC46A3, or an inhibitor of SLC46A2 and SLC46A3) can be mixed with a pharmaceutical carrier, e.g., conventional tableting ingredients such as corn starch, lactose, sucrose, sorbital, talc, stearic acid, magnesium stearate, dicalcium phosphate or gums, and other pharmaceutical diluents, e.g., water, to form a solid preformulation composition containing a homogeneous mixture of an active ingredient. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from 0.1 to about 500 mg of the active ingredient of the present invention. The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coating, such materials including a number of polymeric acids and mixtures of polymeric acids with such materials as shellac, cetyl alcohol, and cellulose acetate.

Suitable surface-active agents include, but are not limited to, non-ionic agents, such as polyoxyethylenesorbitans (e.g., Tween™ 20, 40, 60, 80 or 85) and other sorbitans (e.g., Span™ 20, 40, 60, 80, or 85). Compositions with a surface-active agent can comprise between 0.05% and 5% surface-active agent (e.g., between 0.1% and 2.5%). It will be appreciated that other ingredients may be added, for example mannitol or other pharmaceutically acceptable vehicles, if necessary.

Suitable emulsions can be prepared using commercially available fat emulsions, such as Intralipid™, Liposyn™, Infonutrol™, Lipofundin™, and Lipiphysan™. The active ingredient can be either dissolved in a pre-mixed emulsion composition or alternatively it may be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids or soybean lecithin) and water. It will be appreciated that other ingredients can be added, for example glycerol or glucose, to adjust the tonicity of the emulsion. Suitable emulsions will typically contain up to 20% oil, for example, between 5% and 20%. The fat emulsion can comprise fat droplets between 0.1 and 1.0 μm, e.g., 0.1 and 0.5 μm, and have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some examples, the pharmaceutical compositions are administered by the oral or nasal respiratory route for local or systemic effect.

Compositions in preferably sterile pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, or powder compositions may be administered orally or nasally, from devices which deliver the formulation in an appropriate manner.

### II. Use of Inhibitors for Treating Inflammatory Skin Diseases

Aspects of the present disclosure provide methods for treating inflammatory skin diseases using an inhibitor of SLC46A2 and/or SLC46A2. As used herein, “inflammatory skin disease” refers to any disease of the skin in which immune cells infiltrate the skin and/or engage in aberrant signaling. Inflammatory skin diseases include microbial and non-microbial inflammatory skin diseases. Microbial inflammatory skin diseases refer to inflammatory skin diseases that are caused by infection by a microorganism (e.g., bacteria, yeast, virus, fungi) and non-microbial inflammatory skin diseases refer to diseases that are not caused by infection by a microorganism.

Non-limiting examples of inflammatory skin disease include psoriasis (e.g., plaque psoriasis, nail psoriasis, guttate psoriasis, inverse psoriasis, pustular psoriasis, erythrodermic psoriasis), skin infections (e.g., bacterial infections, yeast infections, viral infections, fungal infections), acne, rosacea, cutaneous lupus erythematosus (e.g., acute lupus erythematosus, subacute lupus erythematosus, chronic lupus erythematosus), lichen planus, cutaneous dermatomyositis, pityriasis rubra pilaris, hidradenitis suppurativa, and dermatitis (e.g., atopic dermatitis (also known as eczema), allergic contact dermatitis, irritant contact dermatitis, nummular dermatitis, dyshidrotic dermatitis, stasis dermatitis, and seborrheic dermatitis).

To practice the method disclosed herein, an effective amount of a composition comprising an inhibitor (e.g., a pharmaceutical composition comprising an inhibitor) can be administered to a subject (e.g., a human patient) having or at risk for having an inflammatory skin disease via a suitable route (e.g., topical administration).

The term “subject” refers to a subject who needs treatment as described herein. In some embodiments, the subject is a human (e.g., a human patient) or a non-human mammal (e.g., cat, dog, horse, cow, goat, or sheep). A human subject who needs treatment can be a human patient having, suspected of having, or at risk for having an inflammatory skin disease, e.g., psoriasis, skin infections, atopic dermatitis, acne, and rosacea. A subject having an inflammatory skin disease can be identified by routine medical examination, e.g., medical examination (e.g., history and physical), or laboratory tests (e.g., biopsy, blood tests). Such a subject can exhibit one or more symptoms associated with an inflammatory skin disease, e.g., rashes, dry skin, itching, burning, soreness, thickened or ridged nail, swollen or stiff joints, or a combination thereof. Alternatively, or in addition to, such a subject can have one or more risk factors for an inflammatory skin disease, e.g., viral infections, family history, stress, smoking or exposure to secondhand smoke, heavy alcohol consumption, certain medications, weather, injury to the skin, or combinations thereof.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or virtually any other reason.

Empirical considerations such as the half-life of an agent will generally contribute to the determination of the dosage. Frequency of administration can be determined and adjusted over the course of therapy, and is generally, but not necessarily, based on treatment and/or suppression and/or amelioration and/or delay of an inflammatory skin disease (e.g., psoriasis, skin infections, atopic dermatitis, acne, and rosacea). Alternatively, sustained continuous release formulations of therapeutic agent may be appropriate. Various formulations and devices for achieving sustained release are known in the art.

In some embodiments, dosages of an inhibitor as described herein can be determined empirically in individuals who have been given one or more administration(s) of the inhibitor. For example, individuals are given incremental dosages of the inhibitor, and an indicator and/or a symptom of an inflammatory skin disease can be followed to assess efficacy of the inhibitor.

Any suitable dosing regimen can be used in methods described herein. In some examples, the dosage regimen depends on the pattern of pharmacokinetic decay that the practitioner wishes to achieve. In some examples, dosing from 0.1 to 100 mg of inhibitor per cm² of surface area of the skin can be used. In some examples, dosing from about 0.001 mg to about 100 mg a day can be used. In some examples, dosing frequency is once every day, once every other day, once every week, or longer. In some examples, dosing frequency is multiple times per day. For repeated administrations over several days or longer, depending on the condition, the treatment can be sustained until a desired suppression of symptoms occurs or until sufficient therapeutic levels are achieved to alleviate an inflammatory skin disease, or a symptom thereof. In some embodiments, dosing regimens (including inhibitor used) can vary over time.

In some embodiments, the appropriate dosage of an inhibitor will depend on the specific inhibitor(s) (or pharmaceutical compositions thereof) used, the type and severity of inflammatory skin disease(s), previous therapy, the patient's clinical history and response to the inhibitor(s), and the discretion of the healthcare practitioner.

As used herein, the term “treating” refers to the application or administration of a composition including one or more active agents to a subject who has an inflammatory skin disease (e.g., psoriasis, skin infections, dermatitis, acne, rosacea, cutaneous lupus erythematosus, lichen planus, cutaneous dermatomyositis, pityriasis rubra pilaris, and hidradenitis suppurativa), a symptom of an inflammatory skin disease, and/or a predisposition toward an inflammatory skin disease, with the purpose to cure, heal, alleviate, relieve, alter, remedy, ameliorate, improve, or affect the inflammatory skin disease, the symptom of the inflammatory skin disease, and/or the predisposition toward the inflammatory skin disease.

Alleviating an inflammatory skin disease includes delaying the development or progression of the disease, and/or reducing disease severity. Alleviating the disease does not necessarily require curative results.

As used herein, “delaying” the development of an inflammatory skin disease (e.g., psoriasis, skin infections, atopic dermatitis, acne, and rosacea) means to defer, hinder, slow, retard, stabilize, and/or postpone progression of the inflammatory skin disease. This delay can be of varying lengths of time, depending on the history of the inflammatory skin disease and/or individuals being treated. A method that “delays” or alleviates the development of an inflammatory skin disease and/or delays the onset of the inflammatory skin disease is a method that reduces probability of developing one or more symptoms of the inflammatory skin disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the inflammatory skin disease. Development of the inflammatory skin disease can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence” of an inflammatory skin disease includes initial onset and/or recurrence.

In some embodiments, the inhibitor of SLC46A2 and/or SLC46A3 is administered to a subject in an amount sufficient to reduce levels of SLC46A2-mediated signaling, SLC46A3-mediated signaling, or both by at least 10% (e.g., at least 20%, at least 30%, at least 40%, at least 50%, at least 60%, at least 70%, at least 80%, at least 90%, or more).

In some embodiments, the inhibitor of SLC46A2 and/or SLC46A3 is administered to a subject in an amount sufficient to reduce levels of SLC46A2 biological activity and/or SLC46A3 biological activity by at least 10% (e.g., at least 20%, at least 30%, at least 40%, at least 50%, at least 60%, at least 70%, at least 80%, at least 90%, or more).

In some embodiments, the inhibitor of SLC46A2 and/or SLC46A3 is administered to a subject in an amount sufficient to reduce levels of SLC46A2 (e.g., SLC46A2 protein and/or nucleic acids) and/or SLC46A3 (e.g., SLC46A3 protein and/or nucleic acids) by at least 10% (e.g., at least 20%, at least 30%, at least 40%, at least 50%, at least 60%, at least 70%, at least 80%, at least 90%, or more).

In some embodiments, the inhibitor of SLC46A2 and/or SLC46A3 is administered to a subject in an amount sufficient to reduce levels of inflammation by at least 10% (e.g., at least 20%, at least 30%, at least 40%, at least 50%, at least 60%, at least 70%, at least 80%, at least 90%, or more).

In some embodiments, the inhibitor of SLC46A2 and/or SLC46A3 is administered to a subject in an amount sufficient to reduce epidermal thickening by at least 10% (e.g., at least 20%, at least 30%, at least 40%, at least 50%, at least 60%, at least 70%, at least 80%, at least 90%, or more).

In some embodiments, the inhibitor of SLC46A2 and/or SLC46A3 is administered to a subject in an amount sufficient to reduce infiltration of immune cells (e.g., leukocytes) by at least 10% (e.g., at least 20%, at least 30%, at least 40%, at least 50%, at least 60%, at least 70%, at least 80%, at least 90%, or more).

In some embodiments, the inhibitor of SLC46A2 and/or SLC46A3 is administered to a subject in an amount sufficient to reduce keratinocyte permeabilization by at least 10% (e.g., at least 20%, at least 30%, at least 40%, at least 50%, at least 60%, at least 70%, at least 80%, at least 90%, or more).

The inhibitors of SLC46A2 and/or SLC46A3 can be administered using any suitable method for achieving delivery of the inhibitor to the subject in need thereof. The route of administration can depend on various factors such as the type of inflammatory skin disease to be treated and the site of the disease. In some embodiments, the inhibitor of SLC46A2 and/or SLC46A3 can be administered topically, nasally, parenterally, buccally, or by inhalation. Parenteral administration includes, but is not limited to, subcutaneous, intracutaneous, intravenous, intramuscular, or intrasynovial injection or infusion techniques.

The particular dosage regimen, e.g., dose, timing, and repetition, used in methods described herein will depend on the particular subject and that subject's medical history.

In some embodiments, more than one inhibitor of SLC46A2 can be administered to a subject in need thereof (e.g., a small molecule inhibitor and a peptide inhibitor are administered to the subject). The inhibitor of SLC46A2 can be the same type or different from each other. At least one, at least two, at least three, at least four, or at least five different inhibitors of SLC46A2 can be co-administered. In such instances, inhibitors of SLC46A2 can have complementary activities that do not adversely affect each other. Inhibitors of SLC46A2 can also be used in conjunction with other agents that serve to enhance and/or complement the effectiveness of the inhibitor.

In some embodiments, more than one inhibitor of SLC46A3 can be administered to a subject in need thereof (e.g., a small molecule inhibitor and a peptide inhibitor are administered to the subject). The inhibitor of SLC46A3 can be the same type or different from each other. At least one, at least two, at least three, at least four, or at least five different inhibitors of SLC46A3 can be co-administered. In such instances, inhibitors of SLC46A3 can have complementary activities that do not adversely affect each other. Inhibitors of SLC46A3 can also be used in conjunction with other agents that serve to enhance and/or complement the effectiveness of the inhibitor.

In some embodiments, an inhibitor of SLC46A2 and an inhibitor of SLC46A3 can be administered to a subject in need thereof.

In some embodiments, the inhibitor SLC46A2 and/or the inhibitor of SLC46A3 is administered one or more times to the subject. Alternatively, or in addition, the inhibitor can be administered as part of a combination therapy comprising an inhibitor of SLC46A2 and/or an inhibitor of SLC46A3 and an additional therapeutic agent.

Any therapeutic agent suitable for treating an inflammatory skin disease can be used as an additional therapeutic agent in methods and/or compositions described herein. Non-limiting examples of additional therapeutic agents include anti-inflammatory agents (e.g., steroids such as corticosteroids)) and immunosuppressants (e.g., methotrexate, cyclosporine). Alternatively, in some embodiments no other agents are used.

The term combination therapy, as used herein, embraces administration of these agents in a sequential manner, that is wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular routes, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent, before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Ethics

All animal studies were performed in compliance with the federal regulations set forth in the Animal Welfare Act (AWA), the recommendations in the Guide for the Care and Use of Laboratory Animals of the National Institutes of Health, and the guidelines of the UMass Medical School Institutional Animal Use and Care Committee. All protocols used in this study were approved by the Institutional Animal Care and Use Committee at the UMass Medical School (Protocol 2056).

### Mice

ES cells harboring Slc46a2 locus targeted with Zen-Ub1 cassette were purchased from Mutant Mouse Resource and Research Center (MMRRC) at University of California at Davis, an NIH-funded strain repository, and were donated to the MMRRC by The KOMP Repository, University of California, Davis; originating from David Valenzuela, George Yancopoulos, Regeneron Pharmaceuticals, Inc (RRID: MMRRC_062453-UCD) [89]. ES cells were used to generate chimeric founder mice by standard microinjection into albino C57BL/6J blastocysts, by the UMMS transgenic mouse core. Chimeras were mated to albino C57Bl/6J mice to identify germline transmission. Heterozygous animals were identified using PCR (see Key Resources Table for oligos). Wildtype (Slc46a2^{+/+}) and mutant (Slc46a2^{-/-}) lines were established from these heterozygous and used in all experiments.

Unless otherwise noted, for in vivo analysis of NOD1 responses in mouse skin, female mice aged 4-6 week were used.

### Keratinocyte's Isolation, Culture and Analysis

Adult (6-8 weeks) mouse keratinocytes were isolated from male or female animals using protocols described previously (57). In brief, tail skin was incubated in 1 mg/ml Dispase II (Roche) overnight at 4° C. The epidermis was physically removed from the dermis with tweezers following Dispase II treatment, subsequently digested with TrypLE (Thermo) for 20 min at room temperature, and keratinocytes were detached by vigorously shaking in EpiLife culture medium and filtered with a 70 μm strainer (Fisher scientific). Isolated cells were seeded at a density of 10⁵ cells/cm² cultured with EpiLife (Gibco) in 12-well plates precoated with coating matrix (Gibco) and used between 3 and 5 days after isolation.

For bioassay, conditioned media from these keratinocytes was injected intraperitoneally and leukocyte recruitment assayed, as below. For preparation of conditioned media, keratinocytes were first stimulated with 8 μM TCT, 30 μM iE-DAP or 20 ng/ml LPS for 1 hour (or left unstimulated). Then cells were washed 3 times with culture medium to remove agonists and cultured further in complete media for 24 hours, when the media was collected and centrifuged (500 g for 15 min at 4° C.).

Keratinocyte media was treated with 1 u/ml proteinase K (Sigma) at 37° C. for 30 min, heated at 95° C. for 10 min, or filtered through a 10 kDa molecular weight cutoff filter (Milipore) and filtrate and retentate fractions collected.

For cytokine ELISAs, primary keratinocytes were either mock-treated or treated with 8 μM TCT or 30 μM iE-DAP for 24 h Culture medium was harvested and centrifuged at 500 g for 15 mins and analyzed using ELISA kits detecting CXCL1 (KC), IL-6, TNF-α, IL-1α and IL-1β (R&D systems) following the manufacturer's protocols. In brief, 96-well ELISA plates were coated with capture antibody overnight at room temperature. The next day, the plate was washed three times with wash buffer and incubated with harvested media (100 μl) for 2 h followed by three washes and then probed with biotin-labelled detection antibody for 2 h. After three washes with wash buffer, plates were incubated with HRP-streptavidin for 20 min at room temperature (RT), developed with TMB (3,3′,5,5″-tetramethylbenzidine) substrate for 20 min at RT, and then stopped with IN sulfuric acid. Plate was analyzed at 450 nm with wavelength correction at 540 nm.

For cell permeabilization assays, 1×10⁵ keratinocytes/well were seeded in a 96-well plate (Denville Scientific Inc.). After reaching 80% confluency, 30 μM iE-DAP was added to culture media with 1 nM Sytox red dye (Thermo) and 0.2 nM hoechst stain (Thermo). Plates were then cultured in a Cytation5 (BioTek) live imaging device and each well was imaged every 30 min for 24 hours. For analysis, Gen5 software were used to count the total number of Hoechst-stained nuclei and the total number of permeabilized cells were quantified as the number of Sytox positive nuclei. The Percentage of Sytox⁺ cells was then calculated and plotted.

For scratch assays, 5×10⁵ cells were seeded in 12-well plates and incubated until reaching 90-95% confluency. A scratch was then made using a 1 ml pipet tip and imaged immediately and 48 h later, on a Nikon upright microscope at 4×. For analysis, the cleared area, resulting from the scratch, was determined by manually demarcating with the SPOT software.

### Dermal Fibroblasts

Mouse dermal fibroblasts were isolated as described herein. In brief, mouse pinnae were incubated on a table shaker (200 rpm) in a collagenase D-pronase solution [2.5 mg/ml collagenase-D (Sigma), 20 mg/ml pronase (Sigma) diluted in in 1M Tris, pH 8.0 and containing 1 μM EDTA (Sigma)] for 90 min at 37° C. Fibroblasts were then isolated by pressing digested skin through a 70 μm cell strainer, and cells were collected and cultured in RPMI 1640 medium with 10% FBS, 50 μM 2-mercaptoethanol, 100 μM asparagine, 2 mM glutamine, 1% penicillin-streptomycin for 2-4 passages before stimulating with immune elicitors, and/or harvesting media or cells.

For cytokine ELISAs from dermal fibroblast, cells were challenged with IL-1α (10 ng/ml) or 30 μM iE-DAP for 24 h, and media was collected and processed for ELISA as described above.

### Ear Injections

10 μl of 30 μM iE DAP or PBS was injected in ventral side of right and left pinnae, respectively. After 3 h, both ears were individually harvested, and stored in ice cold PBS on ice until further processing for flow cytometry staining.

### Sample Processing for Flow Cytometry Staining

Pinnae were cut into small pieces and incubated for 30 min at 37° C. in a solution of 1 mg/ml collagenase (Sigma) in DMEM media containing 10% FBS. Ice-cold FACS buffer (2% FBS in PBS) was added to skin samples to stop the reaction. Then samples were placed on 70 μm cell strainers and ground using a cell strainer pestle to create a single cell suspension. Cells were spun at 180 g for 5 min at 4° C. and resuspended in FACS buffer.

For peritoneal cell extraction, animals were euthanized and cells from peritoneal cavity were isolated by peritoneal lavage (58). Harvested cell suspensions were centrifuged at 300 g for 5 min at 4° C. and resuspended in FACS buffer.

1×10⁶ cells were stained with F4/80-PE/Cy7 (1:200), CD45-alexaflour700 (1:200) and Gr1-PE (1:200) for 30 min on ice in the dark. Samples were washed three times with FACS buffer and analyzed with a Cytek Aurora cytometer. Results were analyzed with FlowJo software (Tree Star, USA).

### Cytospin

FACS sorted cells were centrifuged in 200 μL RPMI 1640 media with 10% FBS onto a microscope slide using a Cytospin Universal 320 (Hettich, Germany) and stained with hematoxylin and eosin (H&E). Images were acquired with an upright Nikon microscope equipped with a Canon A620 camera.

### Histology

Pinnae were harvested 3 h after DAP-muropeptide administration or 7 d after topical application of IMQ and fixed in PBS containing 10% formalin. Paraffin-embedded sections were cut at 0.5 mm, stained with H&E, and imaged by light microscopy.

### RNA Extraction and qRT-PCR

Cells or tissue were lysed in TRIzol (BioRad) followed by RNA extraction as per the manufacture's protocol. In brief, TRIzol lysed samples were mixed with chloroform and spun at 12000 g for 15 min at 4° C. RNA from aqueous phase was precipitated with isopropanol and the RNA pellet was washed with 80% ethanol and resuspended in water. cDNA was prepared from 1 μg RNA using iScript gDNA Clear cDNA Synthesis Kit (BioRad) per the manufacturer's protocol. In brief, 1 μg RNA was incubated with DNase mastermix at 25° C. for 5 min followed by reverse transcription for 20 min at 46° C. cDNA was diluted 1:5 and used directly in qPCR reaction.

Real-time quantitative PCR was performed with 0.4 mM primer, 10 μL iQ SYBR Green Supermix (BioRad), in a final volume of 10 μL on CFX96 real-time system (BioRad). All samples were run in triplicate. GAPDH was used to normalize.

### Isolation of RNA From Mouse Tissues and Cells

Lungs: whole lungs were extracted from euthanized mice and 1 mg lung tissue was homogenized in Trizol using a tissue homogenizer and cDNA was prepared as above.

Spleen: whole spleen was extracted from euthanized mice and a single cell suspension was prepared by passing the spleen through a 70 μm cell strainer with a plunger. Isolated splenocytes were resuspended in Trizol and cDNA was prepared as above.

Gut: Whole gut was extracted from euthanized mice and 1 mg gut tissue was homogenized in Trizol using homogenizer and cDNA was prepared as above.

Dermis and epidermis: Pinnae were extracted from euthanized mice and incubated 1 mg/ml Dispase II (Roche) overnight at 4° C. Epidermis was separated from the dermis with forceps. These tissues were homogenized in Trizol and cDNA was prepared as above.

Bone marrow macrophages: tibia and femur bones were extracted from euthanized mice and crushed using mortar and pestle in DMEM medium. The isolated cell suspension was passed through a 70 μm cell strainer and 2×10⁶ cells/mL were plated in DMEM-F12 media supplemented with recombinant MCSF (25 ng/ml). After 7 days, cells were lysed with TRIzol and cDNA was prepared as above.

Peritoneal macrophages: 2 ml of 3% Brewer thioglycolate medium was injected into the peritoneum to elicit the recruitment of peritoneal macrophages. 4 days later, peritoneal cells were harvested by injecting 5 ml FACS buffer into the peritoneal cavity of euthanized mice and collected back. These cells were then resuspended in TRIzol and cDNA was prepared as above.

Dendritic cells: Pan dendritic cells were isolated from euthanized mice using Pan Dendritic Cell Isolation Kit as per manufacturer protocol (Miltenyi Biotech). cDNA was prepared as described above.

Neutrophils: neutrophils were isolated from bone marrow cells. Marrow cells were isolated as described and neutrophils separated from them using histopaque density gradient (Histopaque 1119: Histopaque 1077 in 1:5 (Sigma). The middle layer containing neutrophils was separated and cells were resuspended in TRIzol and cDNA was prepared as above.

### Immunoprecipitation and Immunoblot Assay

Immunoprecipitation of RIPK2 and immunodetection was performed as described previously (59). In brief, 1×10⁶ keratinocytes were stimulated with iE-DAP (30 μM) and then lysed at indicated time points between 5 and 60 minutes with ice cold lysis buffer (T-PER protein extraction buffer with 1× Halt protease inhibitor), and then centrifuged at 13,000 g for 20 min to remove debris. The concentration of proteins in a sample was estimated by Bradford assay using bovine serum albumin (BSA) as a standard, and for whole cell lysates 50 μg protein was loaded into each lane of an SDS-PAGE gel. 500 μg of whole cell lysate was used to immunoprecipitated RIPK2 with an anti-RIPK2 (1:2000, Thermo) antibody and protein-A agarose beads for 4 h at 4° C. The beads were then washed three times with wash buffer [10 mM Tris-Cl (pH 7.5), 150 mM NaCl, 1 mM imidazole, 2 mM B-ME, 0.1% Triton×100 and a protease inhibitor cocktail]. Finally, pelleted beads were suspended in 2×SDS laemmli sample buffer (Biorad) and boiled for 5 min followed by centrifugation for 5 min, and then samples were separated on 8-12% SDS-PAGE. The gel was then transferred to a PVDF membrane by wet transfer method (200 volt for 1 h) and further processed with standard immunoblotting methods. Proteins were detected by commercial antibodies at the following dilutions: RIPK2 1:2000, phospho-RIPK2 1:1000 and β-actin 1:10000), followed by horseradish peroxidase (HRPO)-conjugated secondary antibody (1:10000). Electrochemiluminescence (ECL) (Millipore) reagent was used for visualization.

### IMQ Model of Psoriasis-Like Dermatitis

The induction of psoriasis-like inflammation on mouse pinnae was done as previously described (60). In brief, mice were treated daily for up to 6 d with 5 mg 5% IMQ cream topically applied on one pinna, while the other pinnae were treated with a similar amount of Vaseline, as vehicle control. Ear thickness was measured daily using a digital caliper. The change in ear thickness over time was reported as the difference related to the first day of topical application. For the bacterial assisted psoriasis model, 5 mg 5% IMQ cream was topically applied on one pinna, while the contralateral pinna was treated with a similar amount of Vaseline for 3 days, and then 10⁷ CFU of C. accolens, in 50% glycerol, was topically applied daily to both pinnae. Ear thickness was measured daily using a digital caliper. C. accolens, originally from ATCC, was provided by J. Kang and grown as described (61).

### Tape Stripping

The epidermal barrier of the dorsal ear pinnae was disrupted by mild tape stripping. A small piece of surgical tape (Transpore surgical tape) was manually applied to the skin and removed, repeated 5 consecutive rounds using a fresh piece of tape for each round. Then, 10⁷ CFU C. accolens or 30 μM iE-DAP in 50% glycerol was applied to one pinna and 50% glycerol was applied to contralateral pinna. After 3 h mice were euthanized, and pinnae were processed as described above.

### Click-iE DAP Preparation and Imaging

iE-DAP with an alkyne handle for click-chemistry reaction was synthesized using a standard 8-step chemical synthesis. All amino acids were purchased from TCI America or Chem-Impex. All other chemical reagents were purchased from Sigma Aldrich, Fisher Scientific, Alfa Aesar, or Oakwood Chemical and used without further purification. NMR solvents were purchased from Cambridge Isotope Laboratories, Inc.

The click-muropeptides were purified by semi-prep C18 HPLC. Prior to use in experiments, the click-muropeptides were purified by semi-prep C18 HPLC and analyzed by NMR & MS. (Thermo QExactive Orbitrap at the Mass Spectroscopy Facility and Bruker AV 400 MHz, AV III 600 MHz NMR at the NMR laboratory, Department of Chemistry and Biochemistry, University of Delaware. The structure of biologically active click-iE-DAP and click-MDP are shown in FIGS. 9A-9B, respectively.

When used to challenge primary keratinocytes, click-iE DAP and MDP were similarly active in inducing cell permeabilization compared to iE-DAP and MDP respectively (data not shown). For visualization, mouse keratinocytes were challenged with click-iE-DAP (30 μM) or click-MDP (20 μM) 37° C. for 30-60 min, cells washed 2× with 1×PBS to remove access of click-muropeptides and fixed with 4% paraformaldehyde in PBS at RT for 10 min. Cells were permeabilized with 1% Triton-X in PBS for 10 min at RT and blocked with 1% BSA in PBS. These permeabilized cells were then incubated in click-reaction conditions (250 μM CuSO4, 35 μM BTAA, 60 μM sodium ascorbate) with 2.5 μM CalFluor 488 Azide at RT for 30 mins. Cells were then washed and mounted on slides, with DAPI containing mounting media. Slides were imaged with a Leica SP8 confocal microscope.

### Nano-Particle Preparation and Challenge

A lipid/cholesterol matrix consisting of 1,2-dioleoyl-sn-glycero-3-phosphocholine (DOPC, Avanti), 1,2-distearoyl-sn-glycero-3-phosphocholine (DSPC, Avanti), 1,2-dioleoyl-sn-glycero-3-phospho-(1′-rac-glycerol) (DOPG, Avanti), cholesterol (Avanti), and 1,2-distearoyl-sn-glycero-3-phosphoethanolamine-methoxyl poly (ethylene glycol) 2000 (DSPE-mPEG 2000, Laysan Bio) was used to formulate iE-DAP-encapsulated nanoparticles (NPs). iE-DAP were loaded into lipid-based NPs as follows.

Briefly, a matrix of DOPC/DSPC/DOPG/cholesterol/DSPE-mPEG at 33.5/33.5/20/10/3 mol % was prepared in chloroform and dry lipid/cholesterol films were allowed to form. Films were rehydrated with iE-DAP (Invivogen) prepared in PBS at 1.2 mg/mL and these samples were vortexed for 30 sec every 10 min for 1 hr at 56° C. to complete this process. Following rehydration, samples were ultrasonicated in alternating 20 sec pulse/10 sec off cycles for 5 min at an amplitude of 20% to form iEDAP-encapsulated lipid-based NPs. Samples were dialyzed for 1 hr following ultrasonication. Dynamic light scattering (DLS) and zeta potential measurements using a Malvern Zetasizer were used to characterize NP size and surface charge, respectively. NPs had an average 43.97 nm hydrodynamic diameter, a polydispersity index (PDI) of 0.158, and a zeta potential of −9.90 mV. Quant-IT assay (Thermo Fisher Scientific) measurements were used to measure average iE-DAP encapsulation at 998.1 μg/mL and an average encapsulation efficiency of 57.1%. To visualize iE-DAP-encapsulated NPs, 0.1 mol % of the fluorescent lipid tracer dye 3,3′-dioctadecyloxacarbocyanine perchlorate (DiO) were added to the lipid/cholesterol films. Mouse keratinocytes were challenged iE-DAP loaded NPs, or control empty NPs, at 37° C. at 10 μg/ml final concentration (˜30 μM iE-DAP), and then monitored for Sytox uptake assay for 24 h, as described above. To visualize NP delivery, cells were washed after 30-60 minutes 2× with PBS to remove access NPs and fixed with 4% paraformaldehyde in PBS at RT for 10 min. Cells were blocked with 1% BSA in PBS, washed 3× with PBS and mounted on slides with DAPI containing mounting media. Slides were then visualized with a Leica SP8 confocal microscope.

### Example 1: Slc46a2 is Required for Neutrophil Recruitment in Response to NOD1 Stimulation in the Mouse Peritoneum and Skin

We previously implicated the SLC46 family of transporters in the delivery of muropeptides to cytosolic innate immune receptors in Drosophila and human cell lines (24). To determine whether SLC46 family members contribute to mammalian responses to muropeptides in vivo, we used a classic assay to monitor NOD1 and NOD2 activities in Slc46a2^{-/-}0 and Slc46a3^{-/-} mice (FIGS. 5A-5E) (37-39). iE-DAP or MDP was injected intraperitoneally (IP) and we measured neutrophil recruitment to the peritoneum after 3 h. Like Nod1^{-/-} mice, Slc46a2^{-/-} mice did not respond to iE-DAP but responded like wild-type to MDP (FIG. 1A). By contrast, Slc46a3^{-/-} mice phenocopied Nod2^{-/-} mice, responding normally to iE-DAP but failing to respond to MDP (FIG. 1A).

Slc46a2 is highly expressed in skin epidermis but not found in many other tissues (FIG. 5F and (40, 41)). As the activity of NOD1 agonists in skin has not been extensively characterized (31), the DAP-muropeptide Tracheal Cytotoxin (TCT), or LPS as a control, was injected intradermally (ID) in the pinnae and neutrophil recruitment to the ear was monitored, between 1 and 24 h (FIGS. 5G-5H); leukocyte recruitment following DAP-muropeptide was rapid and robust, peaking at 3 h. Using this intradermal challenge assay, Slc46a2^{-/-} mice failed to respond to iE-DAP, but responded normally to MDP, phenocopying Nod1^{-/-} mice (FIG. 1B). On the other hand, Slc46a3^{-/-} mice were significantly defective in responding to MDP, like Nod2^{-/-} animals. H&E staining of skin sections confirmed that neutrophils were recruited to the skin in response to DAP-muropeptide challenge a Nod1- and Slc46a2-dependent manner (FIG. 5I). These results, with IP or ID challenge assays, demonstrate that Slc46a2 is selectively required for the NOD1 pathway while Slc46a3 is selectively required for the NOD2 pathway, consistent with the idea that they each transport NOD1 or NOD2 ligands, respectively, into the cytosol.

To further probe the specificity of SLC46A2 in the response to DAP-muropeptides, we compared different NOD1 agonists (TCT, Tri-DAP, iE-DAP, and C12-iE-DAP) following ID challenge. Slc46a2-deficient mice displayed significantly reduced neutrophil recruitment in response to TCT, iE-DAP and Tri-DAP challenge, whereas the response to C12-iE-DAP was not significantly changed, consistent with the acyl tail enabling direct plasma membrane penetration of this molecule (FIG. 1C) (37). Further, topical application of DAP-muropeptides on tapestripped mouse skin also triggered robust Slc46a2- and Nod1-dependent neutrophil recruitment (FIG. 6A). Similarly, tape-stripped skin treated with live C. accolens, a common skin commensal with DAP-type peptidoglycan known to modulate local and systemic immunity (32), resulted in robust neutrophil recruitment in WT skin that was significantly decreased in either the Slc46a2 or Nod1 mutants (FIG. 1D). These results support that SLC46A2 functions in the skin epidermis to deliver DAP-muropeptides to cytosolic NOD1 in keratinocytes, triggering a rapid neutrophilic influx.

To investigate the pathways involved in DAP-muropeptide-triggered Slc46a2/Nod1-dependent neutrophil recruitment to the skin, Pycard- (encoding ASC) and Myd88-deficient mice were analyzed. WT and Pycard-deficient mice showed similar responses to ID DAP-muropeptide challenge, whereas the response was significantly reduced in Myd88 mice, like the Slc46a2-deficient mice (FIG. 1E). Given that the DAP-muropeptide (TCT) used in these assays is LPS and lipopeptide free (42), the MyD88 results suggest that DAP-muropeptide challenge might trigger an IL-1 response. In fact, mice lacking a functional IL-1 Receptor (Il1r1^{-/-}) or deficient for both IL-1α and IL-1β encoding genes (Il1a and Il1b) showed significantly reduced responses to intradermal TCT challenge, similar to Nod1^{-/-} or Slc46a2^{-/-} mice (FIG. 1F). We further dissected the individual role of these cytokines (IL-1α and IL-1β); Il1b-deficient mice did not show any defect in responding to DAP-muropeptide (FIG. 6B). By contrast, Il1a-deficient animals failed to respond to DAP-muropeptide challenge (FIG. 1G). This phenotype was further confirmed by inhibiting 1L-1α using neutralizing antibody (FIG. 6C). Keratinocytes are known for their robust IL-1α expression (43), and these results suggest that SLC46A2-transported DAP-muropeptide and ensuing NOD1 activation induces IL-1α release from keratinocytes, and subsequent recruitment of neutrophils in skin.

### Example 2: Primary Mouse Epidermal Keratinocytes Respond to DAP-Muropeptides Via Slc46a2 and Nod1

To examine IL-1α production, primary mouse keratinocytes, which express Slc46a2 and Nod1 (FIG. 6D), were isolated and cultured ex vivo, stimulated with DAP-muropeptide, and supernatants assayed for cytokine production. As predicted, WT keratinocytes released significant levels of IL-1α 24 h post challenge, whereas IL-1α released by Slc46a2- or Nod1-deficient keratinocytes was significantly reduced compared to WT, and not significantly increased compared to unstimulated cells (FIG. 2A). By contrast, primary dermal fibroblasts did not respond to iE-DAP, consistent with almost no expression of Slc46a2 and Nod1 (FIG. 6E). Further, IP-challenge with conditioned media from DAP-muropeptide stimulated keratinocytes, but not control conditioned media, triggered significant neutrophil recruitment (FIG. 6F and FIG. 2B). However, conditioned media from IL1α-deficient keratinocytes or anti-IL-1α-depleted media from WT keratinocytes produced significantly less neutrophil attracting activity, similar to Slc46a2- or Nod1-deficient keratinocyte media (FIG. 2B and FIG. 6G). Other cytokines, including TNF, IL-6, IL-1β and IL-17 were not induced by iE-DAP challenged keratinocytes and were unchanged in the absence of Slc46a2 or Nod1 (FIG. 6H), while IL-23 was not detected in any condition.

Alarmins, like IL-1α, are released by damaged or dying cells, triggering inflammatory responses in nearby cells (44). To monitor cell damage or death, we next examined DAP-muropeptide triggered keratinocyte permeabilization, quantifying membrane impermeable Sytox uptake in a live cell imaging assay (45, 46). WT keratinocytes were markedly permeabilized in response to DAP-muropeptide, while Slc46a2- and Nod1-deficient keratinocytes were largely protected (FIG. 2C). Interestingly, the pan-caspase inhibitor zVAD-FMK also prevented DAPmuropeptide triggered permeabilization of WT keratinocytes (FIG. 2C). To further probe the role of caspases and pyroptosis in this process, Caspase1- and GasderminD-deficient mice were challenged with ID iE-DAP injection, where they exhibited a strong defect in neutrophil recruitment, similar to Slc46a2^{-/-} animals (FIG. 2D). Keratinocytes from these knockouts also showed significantly decreased permeabilization in response to iE-DAP (FIGS. 2E-2F). A similar phenotype was also observed with dimethyl fumurate (DMF), a potent inhibitor of Gasdermin pore formation (47). All together, these data show that DAP-muropeptide stimulation of primary keratinocytes drives cell permeabilization through a pathway requiring Slc46a2 and Nod1, involving a Caspase-1/Gasdermin D pyroptosis-like process, leading to the release of IL-1α.

### Example 3: DAP-Muropeptide Transport Requires Slc46a2 and is Blocked by Methotrexate

To directly evaluate the role of SLC46A2 in DAP-muropeptide transport, we used two complimentary chemical biological approaches. First, a modified, biologically active alkyne derivative of iE-DAP was synthesized and utilized to visualize uptake of this muropeptide into primary keratinocytes using “click-chemistry” (48). After 60 minutes, iE-DAP was clearly detected within both WT and Nod1^{-/-} cells, but not in Slc46a2-deficient keratinocytes, by confocal microscopy or in cell lysates (FIG. 3A and FIG. 7A, respectively). By contrast, Slc46a2 did not affect the intracellular delivery of click-modified MDP (FIGS. 7A-7B), consistent with data in FIGS. 1A-1G that did not implicate Slc46a2 in the transport of this NOD2 ligand. In an orthogonal approach, fluorescently labeled lipid nanoparticles (NP) were loaded with iE-DAP and used to deliver the iE-DAP into keratinocytes, which bypassed the requirement for Slc46a2 but not Nod1 in inducing membrane permeability (FIG. 3B and FIG. 7C).

SLC46A2 is a paralog of the proton-coupled folate transporter SLC46A1 (˜30% identity), which suggests folates and anti-folates could be a common cargo for all SLC46 family proteins (49, 50). The anti-folate methotrexate (MTX) is a potent anti-inflammatory drug commonly used to treat psoriasis and rheumatoid arthritis, with unclear mechanisms of action (36, 51). This led us to whether MTX competes with DAP-muropeptides for docking to/transport by SLC46A2, which was tested by adding increasing concentrations of MTX in the keratinocytebased iE-DAP assays. In a dose dependent manner, MTX inhibited Slc46a2-dependent DAP-muropeptide triggered keratinocyte permeabilization and IL-1α release, similar to the Slc46a2-deficient keratinocytes (FIG. 3C and FIG. 7D). Further, MTX blocked the cytosolic accumulation of “click”-iE-DAP, similar to competition with unlabeled iE-DAP (FIG. 3D and FIG. 7E) but failed to interfere with NP-mediated iE-DAP delivery (FIG. 3F and FIG. 7F).

### Example 4: Slc46a2^{-/-} and Nod1^{-/-} Mice are Resistant to IMQ-Induced Psoriatic Inflammation

The above results show that SLC46A2 is inhibited by MTX and is required for a skin inflammatory response to C. accolens. Interestingly, MTX is used as a first line treatment for the inflammatory skin disease psoriasis, while Corynebacterium spp. are linked to psoriasis and known to exacerbate psoriasis-like phenotypes in a mouse model (33-36). Therefore, we used the imiquimod (IMQ) model to probe the role of Slc46a2 and Nod1 in psoriatic-like inflammation. With a 7-day course of topical IMQ application, WT mice displayed the expected psoriatic-like inflammation, assayed by enhanced ear thickness and H&E histology, while Slc46a2- and Nod1-deficient mice were markedly resistant (FIGS. 4A-4B and FIG. 8A). Application of IMQ for only 3 days followed by topical application C. accolens similarly drove psoriasis-like inflammation in WT mice while both Slc46a2 and Nod1 deficient animals presented dramatically reduced inflammation (FIG. 4C and FIGS. 8B-8C). Further, topical application of MTX reduced the psoriasis like inflammation in WT skin, to levels similar to the mutant strains, while MTX had no observable effect on the residual inflammation in Slc46a2^{-/-} and Nod1^{-/-} skin (FIG. 4D and FIGS. 8D-8E).

To determine if iE-DAP-triggered inflammatory responses occur in human skin, we first analyzed primary foreskin-derived human keratinocytes. For this experiment, keratinocytes were infected with vesicular stomatitis virus (VSV) as a positive control, as it is known to induce cell permeabilization via a GSDME-dependent pyroptotic pathway (FIG. 4E) (46). By contrast, iE-DAP treatment failed to induce membrane permeabilization in these cells (FIG. 4E). Interestingly, primary human keratinocytes, in standard tissue culture conditions, did not express SLC46A2 (FIG. 4F). When these same keratinocytes are cultured at an air-liquid interface on a bed of collagen-embedded dermal fibroblasts they differentiate into a multilayered squamous epithelium (known as a Human Skin Equivalents (HSE)), with many similarities to human skin (52). In the context of the HSE, differentiated human keratinocytes robustly expressed SLC46A2 and these organoids responded to iE-DAP. Upon treatment of the HSE epidermis, but not the dermis, with 30 μM iE-DAP for 24 h, we observed the induction of CXCL8 in dermal fibroblasts (FIG. 4G and FIG. 8F). Moreover, this response was prevented by concomitant treatment with recombinant IL-1R antagonist (IL-1Ra), strongly implicating IL-1 release by keratinocytes in driving CXCL8 induction in HSE dermal fibroblasts. Likewise, in the mouse system, supernatants from iE-DAP-stimulated primary keratinocytes triggered, via Slc46a2- and Nod1-dependent signaling, CXCL1 (KC) production in dermal fibroblasts (FIG. 8G).

### REFERENCES

1. S. A. Ragland, J. C. Kagan, Cytosolic detection of phagosomal bacteria—Mechanisms underlying PAMP exodus from the phagosome into the cytosol. Mol Microbiol 116, 1420-1432 (2021).
2. V. A. Rathinam, K. A. Fitzgerald, Cytosolic surveillance and antiviral immunity. Curr Opin Virol 1, 455-462 (2011).
3. H. Okude, D. Ori, T. Kawai, Signaling Through Nucleic Acid Sensors and Their Roles in Inflammatory Diseases. Front Immunol 11, 625833 (2020).
4. K. V. Swanson, M. Deng, J. P. Ting, The NLRP3 inflammasome: molecular activation and regulation to therapeutics. Nat Rev Immunol 19, 477-489 (2019).
5. E. L. Johnston, B. Heras, T. A. Kufer, M. Kaparakis-Liaskos, Detection of Bacterial Membrane Vesicles by NOD-Like Receptors. Int J Mol Sci 22, (2021).
6. B. Sundaram, T. D. Kanneganti, Advances in Understanding Activation and Function of the NLRC4 Inflammasome. Int J Mol Sci 22, (2021).
7. S. Bauernfried, M. J. Scherr, A. Pichlmair, K. E. Duderstadt, V. Hornung, Human NLRP1 is a sensor for double-stranded RNA. Science 371, (2021).
8. M. Kudo, K. Kobayashi-Nakamura, N. Kitajima, K. Tsuji-Naito, Alternate expression of PEPT1 and PEPT2 in epidermal differentiation is required for NOD2 immune responses by bacteria-derived muramyl dipeptide. Biochem Biophys Res Commun 522, 151-156 (2020).
9. R. D. Luteijn et al., SLC19A1 transports immunoreactive cyclic dinucleotides. Nature 573, 434-438 (2019).
10. C. Ritchie, A. F. Cordova, G. T. Hess, M. C. Bassik, L. Li, SLC19A1 Is an Importer of the Immunotransmitter cGAMP. Mol Cell 75, 372-381 e375 (2019).
11. W. Strober, T. Watanabe, NOD2, an intracellular innate immune sensor involved in host defense and Crohn's disease. Mucosal Immunol 4, 484-495 (2011).
12. Z. Al Nabhani, G. Dietrich, J. P. Hugot, F. Barreau, Nod2: The intestinal gate keeper. PLoS Pathog 13, e1006177 (2017).
13. K. H. Schleifer, O. Kandler, Peptidoglycan types of bacterial cell walls and their taxonomic implications. Bacteriol Rev 36, 407-477 (1972).
14. T. Mukherjee et al., NOD1 and NOD2 in inflammation, immunity and disease. Arch Biochem Biophys 670, 69-81 (2019).
15. O. Irazoki, S. B. Hernandez, F. Cava, Peptidoglycan Muropeptides: Release, Perception, and Functions as Signaling Molecules. Front Microbiol 10, 500 (2019).
16. M. G. Ismair et al., hPepT1 selectively transports muramyl dipeptide but not Nod1-activating muramyl peptides. Can J Physiol Pharmacol 84, 1313-1319 (2006).
17. G. M. Charriere et al., Identification of Drosophila Yin and PEPT2 as evolutionarily conserved phagosome-associated muramyl dipeptide transporters. J Biol Chem 285, 20147-20154 (2010).
18. J. Lee et al., pH-dependent internalization of muramyl peptides from early endosomes enables Nod1 and Nod2 signaling. J Biol Chem 284, 23818-23829 (2009).
19. N. Nakamura et al., Endosomes are specialized platforms for bacterial sensing and NOD2 signaling. Nature 509, 240-244 (2014).
20. S. Sasawatari et al., The solute carrier family 15A4 regulates TLR9 and NOD1 functions in the innate immune system and promotes colitis in mice. Gastroenterology 140, 1513-1525 (2011).
21. Y. Hu, F. Song, H. Jiang, G. Nunez, D. E. Smith, SLC15A2 and SLC15A4 Mediate the Transport of Bacterially Derived Di/Tripeptides To Enhance the Nucleotide-Binding Oligomerization Domain-Dependent Immune Response in Mouse Bone Marrow-Derived Macrophages. J Immunol 201, 652-662 (2018).
22. L. X. Heinz et al., TASL is the SLC15A4-associated adaptor for IRF5 activation by TLR7-9. Nature 581, 316-322 (2020).
23. I. Rimann et al., The solute carrier SLC15A4 is required for optimal trafficking of nucleic acid-sensing TLRs and ligands to endolysosomes. Proc Natl Acad Sci USA 119, e2200544119 (2022).
24. D. Paik et al., SLC46 Family Transporters Facilitate Cytosolic Innate Immune Recognition of Monomeric Peptidoglycans. J Immunol 199, 263-270 (2017).
25. C. Chen et al., Characterization of the mouse gene, human promoter and human cDNA of TSCOT reveals strong interspecies homology. Biochim Biophys Acta 1493, 159-169 (2000).
26. K. Y. Kim et al., Expression Analyses Revealed Thymic Stromal Co-Transporter/Slc46A2 Is in Stem Cell Populations and Is a Putative Tumor Suppressor. Mol Cells 38, 548-561 (2015).
27. M. Uhlen et al., Proteomics. Tissue-based map of the human proteome. Science 347, 1260419 (2015).
28. P. M. Elias, Skin barrier function. Curr Allergy Asthma Rep 8, 299-305 (2008).
29. J. A. Bouwstra, M. Ponec, The skin barrier in healthy and diseased state. Biochim Biophys Acta 1758, 2080-2095 (2006).
30. K. S. Kobayashi et al., Nod2-dependent regulation of innate and adaptive immunity in the intestinal tract. Science 307, 731-734 (2005).
31. J. Harder, G. Nunez, Functional expression of the intracellular pattern recognition receptor NOD1 in human keratinocytes. J Invest Dermatol 129, 1299-1302 (2009).
32. V. K. Ridaura et al., Contextual control of skin immunity and inflammation by Corynebacterium. J Exp Med 215, 785-799 (2018).
33. A. Balato et al., Human Microbiome: Composition and Role in Inflammatory Skin Diseases. Arch Immunol Ther Exp (Warsz) 67, 1-18 (2019).
34. Z. Stehlikova et al., Crucial Role of Microbiota in Experimental Psoriasis Revealed by a Gnotobiotic Mouse Model. Front Microbiol 10, 236 (2019).
35. P. Zanvit et al., Antibiotics in neonatal life increase murine susceptibility to experimental psoriasis. Nat Commun 6, 8424 (2015).
36. A. K. J. a. M. E. Weinblatt, in Rheumatology, M. H. A. S. E. G. J. S. M. W. M. Weisman, Ed. (Elsevier, Philadelphia PA USA, 2018), vol. 1, chap. 66.
37. J. Masumoto et al., Nod1 acts as an intracellular receptor to stimulate chemokine production and neutrophil recruitment in vivo. J Exp Med 203, 203-213 (2006).
38. H. L. Rosenzweig et al., Activation of NOD2 in vivo induces IL-1beta production in the eye via caspase-1 but results in ocular inflammation independently of IL-1 signaling. J Leukoc Biol 84, 529-536 (2008).
39. J. G. Magalhaes et al., Murine Nod1 but not its human orthologue mediates innate immune detection of tracheal cytotoxin. EMBO Rep 6, 1201-1207 (2005).
40. C. Wu, X. Jin, G. Tsueng, C. Afrasiabi, A. I. Su, BioGPS: building your own mash-up of gene annotations and expression profiles. Nucleic Acids Res 44, D313-316 (2016).
41. M. Karlsson et al., A single-cell type transcriptomics map of human tissues. Sci Adv 7, (2021).
42. T. Kaneko et al., Monomeric and polymeric gram-negative peptidoglycan but not purified LPS stimulate the Drosophila IMD pathway. Immunity 20, 637-649 (2004).
43. M. H. Orzalli et al., An Antiviral Branch of the IL-1 Signaling Pathway Restricts Immune-Evasive Virus Replication. Mol Cell 71, 825-840 e826 (2018).
44. N. C. Di Paolo, D. M. Shayakhmetov, Interleukin 1alpha and the inflammatory process. Nat Immunol 17, 906-913 (2016).
45. P. Orning et al., Pathogen blockade of TAK1 triggers caspase-8-dependent cleavage of gasdermin D and cell death. Science 362, 1064-1069 (2018).
46. M. H. Orzalli et al., Virus-mediated inactivation of anti-apoptotic Bcl-2 family members promotes Gasdermin-E-dependent pyroptosis in barrier epithelial cells. Immunity 54, 1447-1462 e1445 (2021).
47. F. Humphries et al., Succination inactivates gasdermin D and blocks pyroptosis. Science 369, 1633-1637 (2020).
48. P. Shieh et al., CalFluors: A Universal Motif for Fluorogenic Azide Probes across the Visible Spectrum. J Am Chem Soc 137, 7145-7151 (2015).
49. J. L. Parker et al., Structural basis of antifolate recognition and transport by PCFT. Nature 595, 130-134 (2021).
50. R. Zhao, S. Aluri, I. D. Goldman, The proton-coupled folate transporter (PCFT-SLC46A1) and the syndrome of systemic and cerebral folate deficiency of infancy: Hereditary folate malabsorption. Mol Aspects Med 53, 57-72 (2017).
51. A. M. Alqarni, M. P. Zeidler, How does methotrexate work? Biochem Soc Trans 48, 559-567 (2020).
52. M. W. Carlson, A. Alt-Holland, C. Egles, J. A. Garlick, Three-dimensional tissue models of normal and diseased skin. Curr Protoc Cell Biol Chapter 19, Unit 19 19 (2008).
53. P. A. D. Bastos, R. Wheeler, I. G. Boneca, Uptake, recognition and responses to peptidoglycan in the mammalian host. FEMS Microbiol Rev 45, (2021).
54. E. Schrezenmeier, T. Dorner, Mechanisms of action of hydroxychloroquine and chloroquine: implications for rheumatology. Nat Rev Rheumatol 16, 155-166 (2020).
55. B. N. Cronstein, T. M. Aune, Methotrexate and its mechanisms of action in inflammatory arthritis. Nat Rev Rheumatol 16, 145-154 (2020).
56. P. Rider et al., IL-1alpha and IL-1beta recruit different myeloid cells and promote different stages of sterile inflammation. J Immunol 187, 4835-4843 (2011).
57. F. Li, C. A. Adase, L. J. Zhang, Isolation and Culture of Primary Mouse Keratinocytes from Neonatal and Adult Mouse Skin. J Vis Exp, (2017).
58. Ray, A. and B. N. Dittel, Isolation of mouse peritoneal cavity cells. J Vis Exp, (2010).
59. Bharadwaj, R., et al., EhRho1 regulates phagocytosis by modulating actin dynamics through EhFormin1 and EhProfilin1 in Entamoeba histolytica. Cell Microbiol,. 20(9): e12851 (2018).
60. Gray, E. E., et al., Deficiency in IL-17-committed Vgamma4(+) gammadelta T cells in a spontaneous Sox13-mutant CD45.1(+) congenic mouse substrain provides protection from dermatitis. Nat Immunol 14(6): p. 584-92 (2013).
61. Spidale, N. A., et al., Neonatal-derived IL-17 producing dermal gammadelta T cells are required to prevent spontaneous atopic dermatitis. Elife 9 (2020).

### Other Embodiments

As additional description to the embodiments described below, the present disclosure describes the following embodiments.

Embodiment 1 is a method for treating an inflammatory skin disease, the method comprising administering to a subject in need thereof an effective amount of an inhibitor of solute carrier family 46 member 2 (SLC46A2) and/or an inhibitor of solute carrier member 3 (SLC46A3).

Embodiment 2 is the method of embodiment 1, wherein the inhibitor is selected from the group consisting of a small molecule inhibitor, a peptide inhibitor, an antibody or antigen binding fragment thereof, and an agent that inhibits expression of SLC46A2 and/or SLC46A3.

Embodiment 3 is the method of embodiment 2, wherein the agent that inhibits expression of SLC46A2 and/or SLC46A3 is selected from the group consisting of short interfering nucleic acid (siNA), short interfering RNA (siRNA), double-stranded RNA (dsRNA), micro-RNA (miRNA), and short hairpin RNA (shRNA).

Embodiment 4 is the method of any one of embodiments 1-3, wherein the inhibitor is formulated for topical administration.

Embodiment 5 is the method of any one of embodiments 1-4, wherein the inhibitor is formulated in a pharmaceutical composition, which further comprises a pharmaceutically acceptable carrier.

Embodiment 6 is the method of any one of embodiments 1-5, wherein the subject is a human patient having or at risk for having an inflammatory skin disease.

Embodiment 7 is the method of embodiment 6, wherein the inflammatory skin disease is psoriasis, skin infections, dermatitis, acne, rosacea, cutaneous lupus erythematosus, lichen planus, cutaneous dermatomyositis, pityriasis rubra pilaris, and hidradenitis suppurativa.

Embodiment 8 is the method of any one of embodiments 1-7, wherein the inhibitor is administered topically.

Embodiment 9 is the method of any one of embodiments 1-7, wherein the inhibitor is administered systemically.

Embodiment 10 is the method of any one of embodiments 1-9, further comprising administering to the subject an additional therapeutic agent.

Embodiment 11 is the method of claim 10, wherein the additional therapeutic agent is an anti-inflammatory agent or an immunosuppressant.

Embodiment 12 is the method of embodiment 11,wherein the anti-inflammatory agent is methotrexate.

Embodiment 13 is the method of any one of embodiments 10-12, wherein the inhibitor and the additional therapeutic agent are formulated in a single formulation.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating an inflammatory skin disease, the method comprising administering to a subject in need thereof an effective amount of an inhibitor of solute carrier family 46 member 2 (SLC46A2) and/or an inhibitor of solute carrier member 3 (SLC46A3).

2. The method of claim 1, wherein the inhibitor is selected from the group consisting of a small molecule inhibitor, a peptide inhibitor, an antibody or antigen binding fragment thereof, and an agent that inhibits expression of SLC46A2 and/or SLC46A3.

3. The method of claim 2, wherein the agent that inhibits expression of SLC46A2 and/or SLC46A3 is selected from the group consisting of short interfering nucleic acid (siNA), short interfering RNA (siRNA), double-stranded RNA (dsRNA), micro-RNA (miRNA), and short hairpin RNA (shRNA).

4. The method of claim 1, wherein the inhibitor is formulated for topical administration.

5. The method of claim 1, wherein the inhibitor is formulated in a pharmaceutical composition, which further comprises a pharmaceutically acceptable carrier.

6. The method of claim 1, wherein the subject is a human patient having or at risk for having an inflammatory skin disease.

7. The method of claim 6, wherein the inflammatory skin disease is psoriasis, skin infections, dermatitis, acne, rosacea, cutaneous lupus erythematosus, lichen planus, cutaneous dermatomyositis, pityriasis rubra pilaris, and hidradenitis suppurativa.

8. The method of claim 1, wherein the inhibitor is administered topically.

9. The method of claim 1, wherein the inhibitor is administered systemically.

10. The method of claim 1, further comprising administering to the subject an additional therapeutic agent.

11. The method of claim 10, wherein the additional therapeutic agent is an anti-inflammatory agent or an immunosuppressant.

12. The method of claim 11, wherein the anti-inflammatory agent is methotrexate.

13. The method of claim 10, wherein the inhibitor and the additional therapeutic agent are formulated in a single formulation.
